(19) United States
(12) Patent Application Publication
(10) Pub. No.: US 2017/0045494 A1
(43) Pub. Date: Feb. 16, 2017

(54) SYSTEMS AND METHODS FOR IDENTIFYING COAGULOPATHIES

(71) Applicant: T2 Biosystems, Inc., Lexington, MA (US)

(72) Inventors: Thomas Jay LOWERY, JR., Belmont, MA (US); Walter W. MASSEFSKI, JR., Sharon, MA (US); Vyacheslav PAPKOV, Waltham, MA (US); Lynell R. SKEWIS, Brighton, MA (US)

(21) Appl. No.: 15/306,901

(22) PCT Filed: Apr. 27, 2015
(86) PCT No.: PCT/US2015/027784

§ 371 Date: Oct. 26, 2016

Related U.S. Application Data
(60) Provisional application No. 61/985,241, filed on Apr. 28, 2014.

(51) Int. Cl. G01N 33/49; G01N 24/08; G01R 33/44

(52) U.S. Cl. CPC G01N 33/4905; G01R 33/448; G01N 24/08

(57) ABSTRACT

The invention features a diagnostic platform utilizing T2 magnetic resonance to directly measure integrated reactions in whole blood samples such as clotting, clot contraction, and fibrinolysis to provide a comprehensive assessment of hemostatic parameters on a single instrument in minutes. The methods of the invention can be performed with less than 1 mL of blood and minimal sample handling.

### BACKGROUND OF THE INVENTION

Clinical hemostasis involves the controlled rapid transformation of blood flowing under pressure to a highly localized, largely impermeable seal at sites of vascular damage followed by containment and then dissolution of clot formation. These ordered sequential changes in clot structure are required to prevent untoward bleeding in vivo while limiting the risk of thrombotic vascular occlusion.

Thrombosis and bleeding are among the foremost causes of morbidity and mortality. The introduction of novel anticoagulants has increased the need for rapid and accurate assessment of their activities. However, laboratory assessment of hemostasis remains difficult for some common clinical situations. Contemporary clinical laboratory methods are based on measuring components of hemostasis (e.g., prothrombin time, activated partial thromboplastin time, platelet aggregometry) or global function as reflected in mechanical clot strength (e.g., thromboelastography, thromboelastometry). These methods successfully identify many, but not all, bleeding disorders. Additionally, existing methods often provide little insight into the risk of thrombosis; lack sensitivity towards measuring fibrinolytic activity; require complex mechanical instrumentation; and typically require specialized technical expertise not available in most hospital laboratories. Existing methods can require blood draws of 1-25 ml and 30-150 minutes for sample processing and measurement.

There is a clinical need for a diagnostic platform that can measure both individual hemostatic parameters and integrated hemostasis, while eliminating sample modification prior to analysis, producing data output in as little as a few minutes with the option to monitor samples for hours, and reducing volume requirements over existing methodologies.

### SUMMARY OF THE INVENTION

The present invention features methods for detecting a change in a blood sample using time-resolved relaxation time acquisition methodology. The provided methods for measuring hemostasis are simple to practice, rapid, and reliable.

In a first aspect, the invention features a method for monitoring a clotting process in a whole blood sample including: (a) providing uncoagulated whole blood, fibrinogen, and a clotting activation reagent; (b) combining the fibrinogen, the clotting activation reagent, and the uncoagulated whole blood to form a reaction mixture that includes from 50% (v/v) to 90% (v/v) (i.e., 60±10%, 70±10%, 80±10%, or 87.5±2.5% (v/v)) whole blood and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

The invention further features a method for monitoring a clotting process in a platelet rich plasma sample including: (a) providing uncoagulated platelet rich plasma, fibrinogen, and a clotting activation reagent; (b) combining the fibrinogen, the clotting activation reagent, and the uncoagulated platelet rich plasma to form a reaction mixture that includes from 50% (v/v) to 90% (v/v) (i.e., 60±10%, 70±10%, or 80±10% (v/v)) platelet rich plasma and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

The invention also features a method for monitoring a clotting process in a platelet poor plasma sample including: (a) providing uncoagulated platelet poor plasma, fibrinogen, and a clotting activation reagent; (b) combining the fibrinogen, the clotting activation reagent, and the uncoagulated platelet poor plasma to form a reaction mixture that includes from 50% (v/v) to 90% (v/v) (i.e., 60±10%, 70±10%, 80±10%, or 87.5±2.5% (v/v)) platelet poor plasma and a fibrinogen concentration greater than or equal to about 0.5 mg/mL (i.e., the added amount of fibrinogen in the reaction mixture is 0.65±0.15, 0.75±0.15, 0.85±0.15, 0.95±0.15, 1.05±0.15, or 1.25±0.25 mg/mL); (c) making a series of magnetic resonance relaxation rate measurements of water in the reaction mixture; and (d) on the basis of the results of step (c), determining the clotting time.

In particular embodiments of any of the above methods, the method further includes repeating steps (a)-(d) to produce a replicate value of the clotting time (i.e., making measurements in, for example, duplicate or triplicate). A clotting time value for a particular sample and coagulation conditions can be the average of the replicate measurements.

In another embodiment of any of the above methods, the fibrinogen concentration in the reaction mixture is sufficient to produce a clotting time having coefficient of variation of less than 7%, 6%, 5%, 4%, or 3.5% when the clotting time is measured at least 10 times. The methods of the invention can reduce the variability observed in clotting time measurements relative to measurements made on samples to which no fibrinogen has been added.

In a particular embodiment of any of the above methods, step (c) includes making a series of magnetic resonance relaxation rate measurements of water in the reaction mixture within a sample tube having a total volume of from 30 to 60 μL (i.e., 35±10, 45±10, or 55±5 μL).

The invention features a method for monitoring a clotting process in a whole blood sample including: (a) providing uncoagulated whole blood and a clotting activation reagent; (b) combining the clotting activation reagent and the uncoagulated whole blood in a sample tube to form a reaction mixture that includes from 50% (v/v) to 90% (v/v) (i.e., 60±10%, 70±10%, 80±10%, or 87.5±2.5% (v/v)) whole blood and a total volume of from 30 to 60 μL (i.e., 35±10, 45±10, or 55±5 μL); (c) making a series of magnetic resonance relaxation rate measurements of water in the sample tube; and (d) on the basis of the results of step (c), determining the clotting time.

The invention also features a method for monitoring a clotting process in a platelet rich plasma sample including: (a) providing uncoagulated platelet rich plasma and a clotting activation reagent; (b) combining the clotting activation reagent and the uncoagulated platelet rich plasma in a sample tube to form a reaction mixture that includes from 50% (v/v) to 90% (v/v) (i.e., 60±10%, 70±10%, 80±10%, or 87.5±2.5% (v/v)) platelet rich plasma and a total volume of from 30 to 60 μL (i.e., 35±10, 45±10, or 55±5 μL); (c) making a series of magnetic resonance relaxation rate measurements of water in the sample tube; and (d) on the basis of the results of step (c), determining the clotting time.

The invention also features a method for monitoring a clotting process in a platelet poor plasma sample including: (a) providing uncoagulated platelet poor plasma and a clotting activation reagent; (b) combining the clotting activation reagent and the uncoagulated platelet poor plasma in a sample tube to form a reaction mixture that includes from 50% (v/v) to 90% (v/v) (i.e., 60±10%, 70±10%, 80±10%, or 87.5±2.5% (v/v)) platelet poor plasma and a total volume of from 30 to 60 μL (i.e., 35±10, 45±10, or 55±5 μL); (c) making a series of magnetic resonance relaxation rate measurements of water in the sample tube; and (d) on the basis of the results of step (c), determining the clotting time.

In any of the above methods, the fibrinogen and/or the clotting activation reagent can be provided as a solution.

In any of the above methods, the clotting activation reagent can be selected from RF, AA, ADP, CK, TRAP, epinephrine, collagen, tissue factor, celite, ellagic acid, and thrombin, or any other clotting activation agent described herein.

In particular embodiments of any of the above methods, step (c) includes making a series of magnetic resonance relaxation rate measurements of water in the reaction mixture within a sample tube, wherein the inner surface of the sample tube controls fibrin adhesion. The sample tube can be any type of tube or coating described herein for the control of fibrin adhesion.

In any of the above methods, step (c) can include (i) making a plurality of T2 relaxation rate measurements of water in the reaction mixture to produce a plurality of decay curves, and (ii) calculating from the plurality of decay curves a plurality of T2 relaxation spectra.

In a related aspect, the invention features a method of evaluating a blood sample from a subject including (i) performing any one or more of the methods described above on the blood sample, or an extract thereof, to determine the clotting time; and (ii) on the basis of step (i), determining whether the subject is hypercoagulable, hypocoagulable, or normal.

In any of the above methods, step (c) can include determining the fibrinogen level of the blood sample.

In any of the above methods, step (c) can include determining the hematocrit of the blood sample, wherein the blood sample is a whole blood sample.

In any of the above methods, step (c) can include determining the platelet activity of the blood sample, wherein the blood sample is a whole blood sample or platelet rich plasma.

The invention also features a method of evaluating a blood sample from a subject including (i) performing any one or more of the methods described above on the blood sample, or an extract thereof, to determine the clotting time; and (ii) on the basis of step (i), determining whether the subject is at risk of thrombotic complications or the subject is resistant to antiplatelet therapy.

The invention further features a method of evaluating a blood sample from a subject including (i) performing any one or more of the methods described above on the blood sample, or an extract thereof, to determine the clotting time; and (ii) on the basis of step (i), determining whether the subject has a coagulopathy. The coagulopathy can be any coagulopathy described herein.

As used herein, “citrated blood” is blood that has been treated with trisodium citrate (9:1) following standard procedures that minimize platelet activation to prevent coagulation.

As used herein, the term “clotting activation reagent” refers to a clotting initiator or activator. Non-limiting examples include calcium chloride, citrated kaolin, RF, AA, ADP, CK, TRAP, epinephrine, collagen, tissue factor, celite, ellagic acid, and thrombin.

As used herein, the term “coagulopathy” refers to a condition in which the blood's ability to clot (coagulate) is impaired.

As used herein, the term “hypercoagulable” refers to an abnormality of blood coagulation that increases the rate of coagulation and/or extent of coagulability, and may increase the risk of thrombosis.

As used herein, the term “hypocoagulable” refers to an abnormality of blood coagulation that reduces the rate of coagulation and/or extent of coagulability.

As used herein, the term “NMR relaxation rate” refers to any of the following in a sample: T1, T2, T₁ᵣₕₒ, T₂ᵣₕₒ, and T₂*. NMR relaxation rates may be measured and/or represented using T1/T2 hybrid detection methods. Additionally, apparent diffusion coefficient (ADC) can be determined and evaluated (Vidmar et al. NMR in BioMedicine, 2009; and Vidmar et al., Eur J Biophys J. 2008). Additionally, pulsed field gradients with measurement of echo attenuation as a function if the square of gradient strength, Hahn echo sequence, spin echo sequence, FID signal ratios.

As used herein, the term “platelet rich plasma” refers to blood plasma that has been enriched with platelets relative to the whole blood from which it is derived.

As used herein, the term “platelet poor plasma” refers to blood plasma with a very low number of platelets relative to the whole blood from which it is derived. For example, the platelet poor plasma can have less than 10×10³ platelets per microliter of plasma.

As used herein, the term “reader” or “T2reader” refers to a device for detecting coagulation-related activation including clotting and fibrinolysis of samples. T2readers may be used generally to characterize the properties of a sample (e.g., a biological sample such as blood or non-biological samples such as an acrylamide gel). Such a device is described, for example, in International Publication No. WO 2010/051362, which is herein incorporated by reference.

As used herein, the term “resistant to antiplatelet therapy” refers to a weak response, or no response, to an antiplatelet drug in a sample or a subject. For example, resistance to antiplatelet therapy can be monitored by observing platelet function in the presence of an antiplatelet drug, such as an inhibiter of cyclooxygenase 1/thromboxaneA2 receptors (e.g., aspirin), adenosine diphosphate receptors (e.g., clopidogrel), or GPIIb/IIIa receptors (e.g., abciximab, tirofiban).

As used herein, the term “thrombotic complications” refers to complications arising from the formation of thromboses in a subject.

As used herein, the term “whole blood” refers to the blood of a subject that includes red blood cells. Whole blood includes blood which has been altered through a processing step or modified by the addition of an additive (e.g., heparin, citrate, a nanoparticle formulation, fibrinogen, tissue plasminogen activator (TPA), collagen, antithrombotic agents such as abciximab, or other additives).

As used herein, the term “uncoagulated” refers to a whole blood sample, or a fraction thereof, which, upon addition of a clotting activation reagent, is capable of undergoing a coagulation reaction.

Other features and advantages of the invention will be apparent from the following Detailed Description, the drawings, and the claims.

### BRIEF DESCRIPTION OF THE DRAWINGS

FIGS. 1a-1d depict the formation of kinetic spectra by numerical inverse Laplace transform. FIG. 1a demonstrates the fit of an inverse Laplace transform algorithm for relaxation curves at a single point in time of unclotted and clotted blood. FIG. 1b shows a T2 vs. intensity spectra of unclotted and clotted whole blood. FIG. 1c demonstrates the assembly of spectra into a 3D plot to generate a time series of the T2 vs. intensity spectra. FIG. 1d depicts a data simplification wherein the T2 value corresponding to the center of each peak in each T2 spectrum is calculated by averaging over the encompassed T2 values, which are then plotted as a function of time to create T2 relaxation signatures.

FIG. 2 demonstrates dynamic whole blood hemostasis monitoring with T2MR. Clotting was initiated with 3 U/ml thrombin. Part (a) shows a single exchange-averaged water population. Part (b) demonstrates initiation of clot contraction that resolves the serum and erythrocyte water populations. Part (c) demonstrates a steep increase in the upper peak as serum is extruded from the clot. Part (d) shows the completion of contraction and plateau of the upper peak. Part (e) shows the plateau of the middle peak for loosely bound erythrocytes. Part (f) demonstrates a low T2MR signal corresponding to water trapped inside a tightly contracted clot. Fibrinolysis caused by the addition of tPA (30 min) (g) releases erythrocytes back into solution lowering the T2 value, which causes the middle peak to release erythrocytes and decrease in T2 value (h), leaving only (i) the signal associated with the more tightly bound erythrocytes.

FIGS. 3a and 3b represent evaluations of the peak assigned loose clot structure. The sample compartment generating the signal at 300 ms that dropped to 200 ms was assessed by testing two conditions: (3a) re-calcified citrated whole blood activated with thrombin to form a contracted clot, (3b) re-calcified citrated whole blood activated with thrombin followed by addition of tPA. Both samples were mixed with a pipette tip after 190 minutes of incubation.

FIG. 4 shows the comparison between the data collected by T2MR and the Stago ST4 system using Innovin® as an activator for the PT analysis of citrated blood. The best-fit line parameters were slope=1.05, intercept=7.51 and correlation value of 0.97 (R²=0.94).

FIG. 5 shows comparisons of analysis methods for measuring clot strength between T2MR and TEG for activation of citrated blood with calcium and kaolin. The ΔT2 value was calculated by taking the difference between the upper and middle T2 values in the T2MR signature at a time point 13 minutes after activation. Data are shown here using samples from 3 healthy donors where clot strength was adjusted by ex vivo addition of Abcixamab (ReoPro) at a level of 0, 4, or 8 μg/ml prior to measurement. All samples containing Abcixamab were at ΔT2 values of <100 ms or TEG MA values less than 40.

FIGS. 6a and 6b demonstrate the dependence of T2 (ms) and 1/T2 (s⁻¹) on percent hematocrit. (6a) Reconstituted blood samples prepared to span a wide range of hematocrit were measured in triplicate by T2MR to generate a T2 value and in duplicate by the Sysmex pocH-100i hematology analyzer to determine the hematocrit. Measured values (black circles) matched expected values for equation 10 (gray line). (6b) Samples prepared to span a wide range of hematocrit values were measured in triplicate by T2MR to generate a 1/T2 value and in duplicate using a complete blood count analyzer. Measured values (black circles) matched expected values for equation 10 (gray line).

### DETAILED DESCRIPTION

The methods and devices of the invention can be used to assess the risk and occurrence of thrombotic events, including myocardial ischemic events in a patient having or suspected of having vascular disease, particularly in patients who have undergone percutaneous intervention and may be at acute risk of, for example, stent thrombosis, vessel restenosis, myocardial infarction, or stroke. For example, the methods and devices of the invention can be used to assess platelet reactivity (i.e., relative concentration of platelet-associated water molecules in a clot), clotting kinetics, clot strength, clot stability, and time-to-fibrin generation (i.e., R), as indices for risk of a thrombotic event, such as myocardial ischemia, independent of responsiveness to drug therapy (e.g., as assessed by a change in platelet reactivity following administration of an anti-platelet drug such as clopidogrel). These indices can also be used to prevent complications arising from surgical and percutaneous vascular procedures (e.g., stent placement or balloon angioplasty) such as stent thrombosis or re-stenosis. Furthermore, the methods and devices of the invention can be used to identify a safe and effective therapy (e.g., dose, regimen, anti-platelet therapy, among others) for a patient at risk of a thrombotic event or undergoing a surgical procedure.

We have developed and characterized a new diagnostic platform that enables both standard hemostasis measurements as well as measurements that provide novel insights into the dynamics and physical states of blood during clotting and lysis. Assignment of the different T2 Magnetic Resonance (T2MR) signals to distinct blood and clot constituents permits continuous monitoring of the dynamic states of blood components over a wide range of platelet counts, fibrinogen concentrations, hematocrit levels, activator concentrations and other contributors to clotting in whole blood. The T2MR platform allows real-time assessment of the transition of fibrinogen to fibrin polymer, clot contraction by platelets, formation of tightly contracted clots, and fibrinolysis.

Initial correlation and precision studies also demonstrate the potential for clinically relevant measurement of hematocrit, clotting time, platelet reactivity and clot strength with this platform. T2MR may provide novel insights into overall platelet health because clot contraction requires not only the signaling and membrane receptor functions assessed by platelet aggregometry, but also the interaction between the platelet cytoskeleton and fibrin. Preliminary studies suggest that T2MR may show residual platelet capacity to cause clot retraction in whole blood in the presence of inhibitors that block platelet aggregation and may thus find a place in the monitoring of aspirin and other anti-platelet agents to which biological resistance is encountered in the absence of a laboratory correlate.

The T2MR platform combines the flexibility of conforming to standard measurements of hemostasis with analysis of integrated coagulation in whole blood, including the contribution of leukocytes, microparticles and other factors difficult to assess at present. The relative simplicity of the instrumentation and methodology involving a single transfer of whole blood from a test specimen should permit rapid testing requiring no sample preparation and minimal sample volumes.

Lastly, this platform permits rapid and sensitive analysis of whole blood clotting across a spectrum of conditions ranging from impaired hemostasis to hypercoagulable states that cannot be readily assayed using currently available methodology. The unique small sample volume requirement is particularly advantageous for pediatric populations, studies of thrombotic and bleeding disorders in small animal models, and point-of-care testing.

Clotting Initiation

For performing the methods of the invention, clotting may be initiated using a variety of techniques. Citrated kaolin (CK), ellagic acid and celite are common global initiators for aPTT (activated partial thromboplastin time) and whole blood clotting times. For example, to start the clotting process, calcium chloride and kaolin is mixed with a citrated blood sample. CK-activated samples are characterized by clot formations where platelets and fibrin contribute to the clot. Alternatively, an activator RF may be used to initiate clotting with or without the addition to a platelet activator such as TRAP, epinephrine, AA, collagen, or ADP. A-activated samples are characterized by clot formations where fibrin rather than platelets contribute primarily to the clot. Alternatively ADP (or ADP+RF) may be used to activate the clot. ADP-activated samples are characterized by clot formations where fibrin contributes primarily to the clot and platelets contribute to lesser degree. The signal response observed under different activation conditions can be diagnostic of the hemostatic condition of a subject.

Tissue factor is another common global initiator for PT, diluted PT measurements, and extrinsic pathway activation such as that done by EXTEM, a thromboelastometry test. Tissue factor activated samples can lead to clot strength and clot time measurements like CK activated samples.

Other blood clotting activators that can be used in the methods of the invention include collagen, epinephrine, ristocetin, thrombin, calcium, tissue factor, prothrombin, thromboplastin, kaolin, serotonin, platelet activating factor (PAF), thromboxane A2 (TXA2), fibrinogen, von Willebrand factor (VFW), elastin, fibrinonectin, laminin, vitronectin, thrombospondin, and lanthanide ions (e.g., lanthanum, europium, ytterbium, etc.). Combinations of activators can be used, for example, to aid in identifying an underlying hemostatic condition that results in a subject's blood sample being hypocoagulable.

Signal Acquisition and Processing

Standard radiofrequency pulse sequences for the determination of nuclear resonance parameters are known in the art, for example, the Carr-Purcell-Meiboom-Gill (CPMG) is traditionally used if relaxation constant T₂ is to be determined. Optimization of the radiofrequency pulse sequences, including selection of the frequency of the radiofrequency pulses in the sequence, pulse powers and pulse lengths, depends on the system under investigation and is performed using procedures known in the art.

Nuclear magnetic resonance parameters that can be obtained using the methods of the present invention include but are not limited to T1, T2, T1/T2 hybrid, T₁ᵣₕₒ, T₂ᵣₕₒ and T₂*. Typically, at least one of the one or more nuclear resonance parameters that are obtained using the methods of the present invention is spin-spin relaxation constant T2.

As with other diagnostics and analytical instrumentation, the goal of NMR-based diagnostics is to extract information from a sample and deliver a high-confidence result to the user. As the information flows from the sample to the user it typically undergoes several transformations to tailor the information to the specific user. The methods and devices of the invention can be used to obtain diagnostic information about the hemostatic condition of a subject. This is achieved by processing the NMR relaxation signal into one or more series of component signals representative of the different populations of water molecules present, e.g., in a blood sample that is clotting or clotted. For example, NMR relaxation data, such as T2, can be fit to a decaying exponential curve defined by the following equation:

$$f ( t ) = {\sum }_{i = 1}^{n} {A}_{i} exp ( \frac{- t}{T ( i )} ) , \qquad ( 1 )$$

where f(t) is the signal intensity as a function of time, t, Aᵢ is the amplitude coefficient for the ith component, and (T)ᵢ the decay constant (such as T2) for the ith component. For relaxation phenomenon discussed here the detected signal is the sum of a discrete number of components (i=1, 2, 3, 4 . . . n). Such functions are called mono-, bi-, tri-, tetra- or multi-exponential, respectively. Due to the widespread need for analyzing multi-exponential processes in science and engineering, there are several established mathematical methods for rapidly obtaining estimates of Aᵢ, and (T), for each coefficient. Methods that have been successfully applied and may be applied in the processing of the raw data obtained using the methods of the invention include Laplace transforms, algebraic methods, graphical analysis, nonlinear least squares (of which there are many flavors), differentiation methods, the method of modulating functions, integration method, method of moments, rational function approximation, Fadé-Laplace transform, and the maximum entropy method (see Istratov, A. A. & Vyvenko, O. F. Rev. Sci. Inst. 70:1233 (1999)). Other methods, which have been specifically demonstrated for low field NMR include singular value decomposition (Lupu, M. & Todor, D. Chemometrics and Intelligent Laboratory Systems 29:11 (1995)) and factor analysis.

There are several software programs and algorithms available that use one or more of these exponential fitting methods. One of the most widely cited sources for exponential fitting programs are those written and provided by Stephen Provencher, called “DISCRETE” and “CONTIN” (Provencher, S. W. & Vogel, R. H. Math. Biosci. 50:251 (1980); Provencher, S. W. Comp. Phys. Comm. 27:213 (1982)). Discrete is an algorithm for solving for up to nine discrete components in a multi-component exponential curve. CONTIN is an algorithm that uses an Inverse Laplace Transform to solve for samples that have a distribution of relaxation times. Commercial applications using multiexponential analyses use these or similar algorithms. In fact, Bruker minispec uses the publicly-available CONTIN algorithm for some of their analysis. For the invention described here, the relaxation times are expected to be discrete values unique to each sample and not a continuous distribution, therefore programs like CONTIN are not needed although they could be used. The code for many other exponential fitting methods are generally available (Istratov, A. A. & Vyvenko, O. F. Rev. Sci. Inst. 70:1233 (1999)) and can be used to obtain medical diagnostic information according to the methods of the present invention. Information is available regarding how the signal to noise ratio and total sampling time relates to the maximum number of terms that can be determined, the maximum resolution that can be achieved, and the range of decay constants that can be fitted. For a signal to noise ratio of ˜10⁴ the theoretical limit as to the resolution of two decay constants measured, independent of the analytical method, is a resolution δ=(Tᵢ/Tᵢ₊₁) of >1.2 (Istratov, A. A. & Vyvenko, O. F. Rev. Sci. Inst. 70:1233 (1999)). Thus it is believed that the difference between resolvable decay constants scales with their magnitudes, which is not entirely intuitive and is unlike resolution by means of optical detection. The understanding of the maximum resolution and the dependence on resolution on the signal-to-noise ratio will assist in assessing the performance of the fitting algorithm.

The methods of the invention can be compared to systems and devices known in the art, such TEG®, ROTEM®, or SONOCLOT®, or other device to measure a rheological change. Further the methods of the invention can be used on a benchtop NMR relaxometer, benchtop time domain system, or NMR analyzer (e.g., ACT, Bruker, CEM Corporation, Exstrom Laboratories, Quantum Magnetics, GE Security division, Halliburton, HTS-111 Magnetic Solutions, MR Resources, NanoMR, NMR Petrophysics, Oxford Instruments, Process NMR Associates, Qualion NMR Analyzers, SPINLOCK Magnetic Resonance Solutions, or Stelar, Resonance Systems).

The CPMG pulse sequence used to collect data with a T2reader is designed to detect the inherent T2 relaxation time of the sample. Typically, this is dictated by one value, but for samples containing a complex mixture of states (e.g., a sample undergoing a clotting process or dissolution process), a distribution of T2 values can be observed. In this situation, the signal obtained with a CPMG sequence is a sum of exponentials. One solution for extracting relaxation information from a T2reader output is to fit a sum of exponentials in a least-squares fashion. Practically, this requires a priori information on how many functions to fit. A second solution is to use the Inverse Laplace Transform (ILT) to solve for a distribution of T2 values that make up the exponential signal observed. Again, the results of the CPMG sequence S(t), is assumed to be the sum of exponentials:

$$S ( t ) = {\sum }_{t} {A}_{i} {e}^{- t / T {2}_{i}} , \qquad ( 2 )$$

where Aᵢ is the amplitude corresponding to the relaxation time constant T2ᵢ. If, instead of a discrete sum of exponentials, the signal is assumed to be a distribution of T2 values, the sum over states can be represented b:

S(t)=∫₀^{∞}A(1/T2)e^{-t/T2}d(1/T2) (3)

This has the same functional form as the ILT:

F(t)∫₀^{∞}A(s)e^{-st}ds=(4),

and can be treated as such. The ILT of an exponential function requires constraints to solve. A few methods that can be used to impose constraints are CONTIN, finite mixture modeling (FMM), and neural networks (NN). An Inverse Laplace Transform may also be used in the generation of a 3D data set. A 3D data set can be generated by collecting a time series of T2 decay curves and applying an Inverse Laplace Transform to each decay curve to form a 3D data set. Alternatively, a 2D Inverse Laplace Transform can be applied to a pre-assembled 3D data set to generate a transformed 3D data set describing the distribution of T2 times.

In a heterogeneous environment containing two phases, several different exchange regimes may be operative. In such an environment having two water populations (a and b), rₐ and r_{b} correspond to the relaxation rates of water in the two populations; fₐ and f_{b} correspond to the fraction of nuclei in each phase; rₐ and T_{b} correspond to residence time in each phase; and a=(1/τₐ)+(1/τ_{b}) corresponds to the chemical exchange rate. The exchange regimes can be designated as: (1) slow exchange: if the two populations are static or exchanging slowly relative to the relaxation rates rₐ and r_{b}, the signal contains two separate components, decaying with time constants T₂ₐ and T_{2b}; (2) fast exchange: if the rate for water molecules exchanging between the two environments is rapid compared to rₐ and r_{b}, the total population follows a single exponential decay with an average relaxation rate (rₐᵥ) given by the weighted sum of the relaxation rates of the separate populations; and (3) intermediate exchange: in the general case where there are two relaxation rates r₁ and r₂ with r₁ equal to rₐ in the slow exchange limit rₐ<r_{b}, Amp₁+Amp₂=1, and where r_{1,2} goes to the average relaxation rate in the fast exchange limit, the following equations may be applied:

r₁=(½)(rₐ+T_{b}+a)−(½)\sqrt{(r_{b}−rₐ+a)²−4af_{b}(r_{b}−rₐ)} (5)

r₂=(½)(rₐ+r_{b}+a)+(½)\sqrt{(r_{b}−rₐ+)²−4af_{b}(r_{b}−rₐ)} (6)

$$\begin{matrix}{Amp}_{1} = \frac{{r}_{2} - {r}_{av}}{{r}_{2} - {r}_{1}} & ( 7 ) \\ {Amp}_{2} = \frac{{r}_{av} - {r}_{1}}{{r}_{2} - {r}_{1}} & ( 8 )\end{matrix}$$

The invention also features the use of a pulsed field gradient or a fixed field gradient in the collection of relaxation rate data. The invention further features the use of the techniques of diffusion-weighted imaging (DWI) as described in Vidmar et al. (Vidmar et al., NMR Biomed. 23: 34-40 (2010)), which is herein incorporated by reference, or any methods used in porous media NMR (see, e.g., Bergman et al., Phys. Rev. E 51: 3393-3400 (1995), which is herein incorporated by reference).

Other systems can be used to practice the invention, including High resolution benchtop NMR magnets and spectrometers (e.g. Magritek's ultra-compact spectromter, picospin45, NanalysisNMReady 60 p cover the range of 40 MHz-60 MHz), high resolution cryogenic systems, and magnetic resonance imaging systems. With sufficient magnetic field homogeneity, NMR spectroscopy can be used to monitor the chemical shift of more than one water population in a blood sample during clotting. Using this method, unique chemical shift signals can be associated with a tightly bound clot. The different chemical shifts of clot and non-clot signals arise from inherent chemical shifts of nuclei, slowing of water diffusion within a tightly bound clot, as well as microscopic in homogeneities due to paramagnetic centers in heme within red blood cells. The paramagnetic effect has been shown to induce changes in chemical shift be several reports, as known in the art; such as the Evans NMR method and others (see Chu et al., Magn Reson Med, 13:239 (1990).

Alternatively, when the methods of the invention are carried out using the measurement of the T2*, or free induction decay, rather than T2, the relaxation properties of a specific class of, for example, water protons in the sample can be made using an off resonance radiation (i.e., radiation that is not precisely at the Larmour precession frequency). The output can be in the form of the height of a single echo obtained with a T2 measuring pulse sequence rather than a complete echo train. In contrast, normal T2 measurements utilize the declining height of a number of echoes to determine T2. The T2* approach can include the steps of shifting the frequency or strength of the applied magnetic field, and measuring the broadness of the water proton absorption peak, where broader peaks or energy absorption are correlated with higher values of T2. The methods can be carried out using techniques for measuring water diffusion, or utilizing the slope of an echo train. In particular embodiments the measurement is made using a CPMG sequence, or a portion thereof, for example, to remove signals associated with a sample holder.

Database of Signature Curves

In one embodiment, the invention features data processing tools to transform the raw relaxation NMR data into a format that provides signature curves characteristic of hemostatic conditions. Preferred transforms include the Laplace or Inverse Laplace Transform (ILT). The data for each T2 measurement may be transformed from the time dimension where signal intensity is plotted verses time to a “T2 relaxation” dimension. The ILT provides not only information about the different relaxation rates present in the sample and their relative magnitudes but also reports on the breadth of distribution of those signals.

Each acquired T2 relaxation curve has a corresponding two dimensional signature that maps all of the different populations of water, or different T2 relaxation environments, that water is experiencing in the sample. These curves can be compiled to form a 3D data set by stacking the plots over the duration of the clotting time dimension. This can be used to generate a 3D surface that shows how the different populations of water change as a function of time.

The T2 signatures may become clinically relevant in cases whereby underlying pathology is not discriminated by current techniques. For example, patients that have abnormal PT or aPTT values are often worked up with additional studies that includes PT, aPTT, or PT and aPTT analysis using a 1:1 mixture of a patient blood with normal plasma (to rule out a factor deficiency), and the results may point to a specific factor or von Willebrand factor deficiency. However, frequently patients having a clotting factor deficiency have more than one deficiency or have an unbalance or unchecked clotting cascade. In these patients, a single test for one factor deficiency will not reveal the full dysfunction and the clinician must rely on clinical symptomology (excessive bleeding or clotting) and, unfortunately, time may lead to a deleterious outcome. The ability to detect T2 signatures (for patients having normal or abnormal hemostatic conditions) will allow for rapid understanding of complex pathophysiological coagulation cascade conditions and improve clinical outcomes.

Management of Patients

The methods and the devices of the invention can be used to provide a point-of-care evaluation of the hemostatic condition of a patient (e.g., for coagulation management of patients undergoing surgery, to identify patients at risk of thrombotic complications, to identify a patient resistant to antiplatelet therapy, to monitor anticoagulation therapy in a patient, to monitor antiplatelet therapy in a patient, and/or to monitor procoagulant therapy in a patient, for identification of abnormal coagulopathies associated with trauma such as trauma induced coagulopathy, acute coagulopathy; such measurements can be used to inform transfusion decisions).

There are medical circumstances for which a coagulation test is requested including: 1) finding a cause for abnormal bleeding or bruising, 2) in patients with an autoimmune disease, 3) in patients with an underlying cardiovascular disorder, 4) before procedures or surgeries where too much bleeding may be a concern, 5) monitoring anti-coagulant therapy, 6) monitoring peri-operative and trauma patients, and 7) identifying patients with sepsis or septic shock.

Coagulation management of patients undergoing cardiac surgery is complex because of a balance between anticoagulation for cardiopulmonary bypass (CPB) and hemostasis after CPB. Furthermore, an increasing number of patients have impaired platelet function at baseline due to administration of antiplatelet drugs. During CPB, optimal anticoagulation dictates that coagulation is antagonized and platelets are prevented from activation so that clots do not form. After surgery, coagulation abnormalities, platelet dysfunction, and fibrinolysis can occur, creating a situation whereby hemostatic integrity must be restored. The complex process of anticoagulation with heparin, antagonism with protamine, and postoperative hemostasis therapy can be guided by the method and devices of the invention (a point of care test) that assess hemostatic function in a timely and accurate manner.

Problems associated with poor liver function (e.g., decreased synthesis and clearance of clotting factors and platelet defects) can lead to impaired hemostasis and hyperfibrinolysis. Systemic complications, such as sepsis and disseminated intravascular coagulation, further complicate a preexisting coagulopathy. Marked changes in hemostasis in orthotopic liver transplantation occur during the anhepatic phase and immediately after organ reperfusion, mainly a hyperfibrinolysis resulting from accumulation of tissue plasminogen activator due to inadequate hepatic clearance and a release of exogenous heparin and endogenous heparin-like substances. Thus, patients undergoing hepatic surgery, and particularly orthotopic liver transplantation, may have large derangement in their coagulation, making the method and devices of the invention useful for monitoring this patient population.

The method and devices of the invention can be used to guide heparin therapy, among other anticoagulation therapies. For example, the methods of the invention can be carried out with heparinase to assess the coagulation status in the absence of the anticoagulatory effects of heparin. Further, the methods of the invention can be utilized to assess protamine therapy, i.e. to monitor coagulation after protamine therapy and to treat a heparin or protamine induced hemostatic condition. Similarly, analysis could be done pre- and post surgery to determine the anticoagulant or hemostatic status of a surgical patient.

The method and devices of the invention can also be used to guide antiplatelet therapies and identify resistance to antiplatelet therapies. Antiplatelet therapy is increasingly being prescribed for primary and secondary prevention of cardiovascular disease to decrease the incidence of acute cerebro- and cardiovascular events. Antiplatelet drugs typically target to inhibit cyclooxygenase 1/thromboxaneA2 receptors (e.g., aspirin), adenosine diphosphate receptors (e.g., clopidogrel), or GPIIb/IIIa receptors (e.g., abciximab, tirofiban). Although antiplatelet drugs are thought to work primarily by decreasing platelet aggregation, they also have been shown to function as anticoagulants. Because platelets play a key role in overall coagulation, the assessment of the platelet function (more than their number) is critical in the perioperative setting.

The method and devices of the invention can also be used to monitor and/or guide anticoagulant therapies. Anticoagulant therapies (e.g., rivaroxaban, dabigatran, among others) can be monitored for efficacy and compliance, and to ensure avoidance of adverse side effects and/or adverse events (e.g., bleeding events). Dosing adjustments for such therapies have been reported to control bleeding in large, randomized studies. Specifically, dosing of anticoagulants, including direct Factor Xa inhibitors can be used to assist maintenance of a therapeutic window and lead to a reduction of risk of stroke in atrial fibrillation and deep vein thrombosis in patients.

The method and devices of the invention can be used to identify patients resistant to anticoagulant therapy. Anticoagulant therapies include aspirin, plavix, and prasugrel, among other anticoagulants. The method includes (i) administering the anticoagulation therapy to the subject; (ii) evaluating the hemostatic condition of the subject using a method of the invention; and (iii) if the subject is found to be prothrombotic, identifying the subject as a non-responder to the anticoagulation therapy. The identification of non-responders can permit a physician to identify a safe and efficacious anticoagulant to which the patient is responsive, thereby reducing the risk of adverse events (i.e., thrombi formation and stroke).

The method and devices of the invention can be used to monitor procoagulant therapy. The modern practice of coagulation management is based on the concept of specific component therapy and requires rapid diagnosis and monitoring of the pro-coagulant therapy. It has been shown, for example, that platelet transfusion in the perioperative period of coronary artery bypass graft surgery is associated with increased risk for serious adverse events. Clinical judgment alone may not predict who will benefit from a platelet transfusion in the acute perioperative setting. Accordingly, the transfusion of coagulation products should be preferably guided by a point of care test, such as the test provided by the method and devices of the invention.

The method and the devices of the invention can be used to provide a companion diagnostic analysis or test to monitor the effects of a therapeutic compound in a clinical trial or in medical use. The diagnostic analysis may include determining whether or not the subject of the trial or the patient responds to therapy or does not respond to therapy.

The method and the devices of the invention can be used to determine the perfusion through clots, hypercoagulation, hyperclotting, or clotting that is deleterious in a human, as in stroke or cardiac arrest.

The method and the devices of the invention can be used as part of a panel of analyses. The panel can include (i) an immunoassay to proteins that are involved in the coagulation cascade; (ii) an immunoassay to detect fibrin degradation products; (iii) an immune assay to detect antiphospholipid antibodies; (iv) an assay to detect heparin or warfarin or other anticoagulant to assess therapeutic concentration; (v) a PT or aPTT or PTT assay that monitors the plasma prothrombin time; (vi) a genetic test to assess the polymorphic differences in genes encoding proteins that are relevant to (a) the formation or dissolution of thrombin, (b) the coagulation cascade, (c) heparin binding, or (d) therapeutic activity.

The methods and the devices of the invention can be used to manage medical devices with implications towards coagulopathies. An example is a ventricle assist device often used as a bridge for patients awaiting a heart transplant. Patients with such an implant may have clot formation within and outside of the device as a result of the function of the device, and these clots may cause a stroke or another thrombus related event. It may also lead towards infections and bleeding events. A way to avoid these issues is to monitor multiple diagnostic markers that impact the success of the device. For instance, routine testing of PT-INR would allow tighter monitoring of the patients coagulation state, thus, providing tight control of bleeding and clotting events.

The INR is the ratio of a patient's prothrombin time to a normal (control) sample, raised to the power of the International Sensitivity Index value for the analytical system used. A high INR level (e.g., INR=5) indicates that there is a high chance of bleeding, whereas if the INR=0.5 then there is a high chance of having a clot. Normal INR range for a healthy person is 0.9-1.3. For people on warfarin therapy the INR range is typically 2.0-3.0. The target INR may be higher in particular situations, such as for those with a mechanical heart valve, or bridging warfarin with a low-molecular weight heparin (such as enoxaparin (Lovenox)) perioperatively.

Monitoring platelet function, fibrinolysis, clot strength and other factors are equally important in improving outcomes. Understanding the physiologic concentration or activities of these factors are important not just for their interplay with the device, but because they are modulated by the many different therapies often prescribed to patients on these devices (aspirin, rivaroxaban, plavix, warfarin, among others). Another measure that is used with these types of devices is hematocrit, which is often used to adjust the functioning of the device (speed, intensity, etc.) to maintain the function of the heart. The methods and the devices of the invention can provide all of these results (hematocrit, platelet, PT, PT-INR, etc.), potentially simultaneously, and it may provide additional information with respect to clot formation and dissolution. The standard measures above may be combined into an index or signature that identifies the status of the patient and efficacy of the device.

The methods and the devices of the invention can be utilized and configured in multiple ways. They can be used as a laboratory device (e.g., in a central laboratory or STAT laboratory), point-of-care system, or even an implantable monitoring system. For example, as an implantable monitoring system, the sample can consist of continually monitored blood; a vacutainer with whole blood, serum, or plasma; or a finger stick, among other sample fluids.

For example, the methods and the devices of the invention can be utilized for monitoring peri-operative and trauma patients (e.g., providing measures or surrogate measures for PT/INR, aPTT, ACT, Hct, platelet activity, and fibrinolysis). There is a need with these patient populations to quickly and efficiently determine if a transfusion is needed as the patients can exhibit an approximately 6-fold increase in mortality, ischemic events, infection, early onset of complications, and increased ICU/hospital stays. Specifically, determination of the root cause of bleeding events (coagulation cascade vs. platelet activation) can lead to prompt and focused therapy (i.e., transfusion management, anticoagulation monitoring, antiplatelet reactivity, and/or predicting thrombosis risk, among others).

Regardless of the context in which the methods and the devices of the invention are utilized, that the methods of the invention can be used to rapidly measure small volumes is particularly important for platelet function, which previously were difficult to measure using other systems due to the initiation of clotting at the site of the blood draw.

The Clotting Mechanism

For clotting to occur there must be activation of coagulation cascade culminating in fibrin deposition through the action of thrombin on fibrinogen. The coagulation system is composed of a proteolytic cascade that amplifies an initial stimulus with an elegant feedback regulation mechanism to keep the overall process in check and balance. There are two interconnected routes of clotting activation: (i) contact activation (intrinsic pathway); and (ii) tissue factor activation (extrinsic pathway). Both pathways rely on a variety of coagulation factors. Prothrombin is coagulation factor II, thrombin is coagulation factor IIa, fibrinogen is coagulation factor I, and fibrin is coagulation factor Ia. In addition to the coagulation factors, platelets are critical both for the induction and formation of an adequate blood clot. Platelets act as a phospholipid surface upon which prothrombinase complexes are formed and act as a physical scaffold for the developing clot.

The intrinsic coagulation cascade pathway is normally activated by contact with collagen from damaged blood vessels, but many negatively charged surfaces can stimulate this pathway. The intrinsic pathway normally requires platelet activation in order to assemble a tenase complex involving factors VIIIa, IXa, and X. The activation process is linked to the inositol triphosphate (IP3) pathway and involves degranulation and myosin 1 c kinase activation in order to change the platelet shape to ultimately allow adherence.

Clotting may alternatively be activated via the extrinsic coagulation cascade pathway which requires a tissue factor from the surface of extravascular cells. The extrinsic pathway involves complex formation of coagulation factors V, VII, and X. The chief inducer of coagulation in vivo is Tissue Factor (TF), a 47 kDa glycoprotein. The only cells capable of expressing TF in the bloodstream are endothelial cells and monocytes. By contrast, many cells outside the bloodstream, including adventitial fibroblasts, constitutively express TF and thus form an “extravascular envelope” capable of initiating coagulation in the event of a disruption in vascular integrity.

The final stages of the cascade are common to both pathways which involves a tenase complex, the activating complex. Tenase is a contraction of “ten” and the suffix “-ase”, signifying that the complex activates its substrate (inactive factor X) by cleaving it. Intrinsic tenase complex contains the active factor IX (IXa), its cofactor factor VIII (VIIIa), the substrate (factor X), and they are activated by negatively charged surfaces (such as glass, active platelet membrane, sometimes cell membrane of monocytes, or red blood cell membranes). Extrinsic tenase complex is made up of tissue factor, factor VII, the substrate (factor X) and Ca²⁺ as an activating ion.

Activation of factor X, to factor Xa, through either the extrinsic or the intrinsic pathway, leads to the proteolytic conversion of prothrombin to thrombin which, in turn, activates the initiation of the formation of a clot and activates platelets. Factor VIII then catalyzes a transglutaminase reaction to crosslink the fibrin monomers to form a crosslinked network.

The crosslinked fibrin multimers in a clot are broken down to soluble polypeptides by plasmin, a serine protease. Plasmin can be generated from its inactive precursor plasminogen and recruited to the site of a fibrin clot in two ways, by interaction with tissue plasminogen activator at the surface of a fibrin clot, and by interaction with urokinase plasminogen activator at a cell surface. The first mechanism appears to be the major one responsible for the dissolution of clots within blood vessels. The second, although capable of mediating clot dissolution, may normally play a major role in tissue remodeling, cell migration, and inflammation.

Clot dissolution is regulated in two ways. First, efficient plasmin activation and fibrinolysis occur only in complexes formed at the clot surface or on a cell membrane; proteins free in the blood are inefficient catalysts and are rapidly inactivated. Second, both plasminogen activators and plasmin itself are inactivated by specific serpins, proteins that bind to serine proteases to form stable, enzymatically inactive complexes. Pharmacologically, the clot buster tissue plasminogen activator (TPA) and streptokinase or urokinase are used to activate this internal fibrinolytic mechanism.

Medical Conditions

The methods and the device of the invention as herein described may be used for the detection of rheological changes of various liquids, in particular blood samples, for the diagnosis of coagulation, thrombotic disorders, and thrombotic disorders as a result of disease, e.g., sepsis and disseminated intravascular coagulation (DIC), Hemophilia A, Hemophilia B, Hemophilia C, Congenital deficiency of other clotting factors Factor XIII deficiency, Von Willebrand's disease, hemorrhagic disorder due to intrinsic anticoagulants, defibrination syndrome, acquired coagulation factor deficiency, coagulation defects, other, purpura and other hemorrhagic conditions, allergic purpura, Henoch-SchOnlein purpura, thrombocytopenia, immune thrombocytopenic purpura, idiopathic thrombocytopenic purpura, secondary thrombocytopenia, sickle cell anemia, and non-specific hemorrhagic conditions.

The cardiovascular system requires tightly regulated hemostasis. Excessive clotting may cause venous or arterial obstructions, while failure to clot may cause excessive bleeding; both conditions lead to deleterious clinical situations. In most human subjects, the clotting balance is more or less static. However, there are many different clinical parameters (such as hereditary disorders, disease states, therapeutic drugs, or pharmacological stressors) that can alter hemostasis and lead to cardiovascular malfunction.

There are many different known coagulation disorders that are a result of non-functional clotting factors, such as hemophilia (factors VIII (hemophilia A), IX (hemophilia B), XI (hemophilia C)), Alexander disease (factor VII deficiency), prothrombin deficiency (factor II deficiency), Owren's disease (factor V deficiency), Stuart-Prower deficiency (factor X deficiency), Hageman factor deficiency (factor XII deficiency), fibrinogen deficiency (factor I deficiency), and von Willebrand's disease.

The activation of the coagulation cascades appears to be an essential component in the development of multi-organ failure that occurs in end-stage sepsis. Current therapies for sepsis specifically target these cascades for modulation of the progression of the end stages and to prevent organ failure.

The methods and devices of the invention may be used to determine the hematocrit of a blood sample. The hematocrit is a measure of the percent volume occupied by red blood cells in a subject's blood, with normal values for healthy women and men being approximately 36-44% and 41-50%, respectively. The hematocrit depends on both the number of red blood cells in a sample and the size of the red blood cells. The measurement of hematocrit may be useful in establishing a variety of physiological conditions in a subject. Thus, the methods of the invention may be used in the diagnosis of any condition associated with a lower than normal hematocrit or a higher than normal hematocrit. A lower than normal hematocrit may be indicative of anemia, sickle cell anemia, internal bleeding, loss of red blood cells, malnutrition, nutritional deficiencies (e.g., iron, vitamin B12, or folate deficiencies), or over hydration. A higher than normal hematocrit may be indicative of congenital heart disease, dehydration, erythrocytosis, pulmonary fibrosis, polycythemia rubra vera, or abuse of the drug erythropoietin.

The methods of the invention can be used to monitor factors and related coagulopathies associated with disease, disorder or dysfunction such as cancer, autoimmune disorders, lupus erythematosus, Crohn's disease, multiple sclerosis, amyotrophic lateral sclerosis, deep vein or arterial thrombosis, obesity, rheumatoid arthritis, Alzheimer's disease, diabetes, cardiovascular disease, congestive heart failure, myocardial infarction, coronary artery disease, endocarditis, stroke, emboli, pneumonia, ulcerative colitis, inflammatory bowel disease, chronic obstructive pulmonary disease, asthma, infections, transplant recipients, liver disease, hepatitis, pancreas disease and disorders, renal disease and disorders, endocrine disease and disorders, obesity, diseases or disorders associated with thrombocytopenia, and medical (stents, implants, major surgery, joint replacements, pregnancy) or therapeutic (cancer chemotherapy) induced coagulopathy/ies, and risk factors such as heavy smoking, heavy alcohol consumption, sedentary lifestyle. The methods of the invention may also be used to evaluate genomic and proteomic changes that affect coagulation and blood properties.

The methods of the invention can also be used to monitor patients being undergoing anti-coagulant and/or anti-platelet therapy. Examples of anti-thrombotics (e.g., thrombolytics, anticoagulants, and antiplatelet drugs) that can be monitored using the methods of the invention include, without limitation, vitamin K antagonists such as acenocoumarol, clorindione, dicumarol, diphenadione, ethyl biscoumacetate, phenprocoumon, phenindione, tioclomarol, and warfarin; heparin group (platelet aggregation inhibitors) such as antithrombin III, bemiparin, dalteparin, danaparoid, enoxaparin, heparin, nadroparin, parnaparin, reviparin, sulodexide, and tinzaparin; other platelet aggregation inhibitors such as abciximab, acetylsalicylic acid (aspirin), aloxiprin, beraprost, ditazole, carbasalate calcium, cloricromen, clopidogrel, dipyridamole, epoprostenol, eptifibatide, indobufen, iloprost, picotamide, prasugrel, ticlopidine, tirofiban, treprostinil, and triflusal; enzymes such as alteplase, ancrod, anistreplase, brinase, drotrecogin alfa, fibrinolysin, procein C, reteplase, saruplase, streptokinase, tenecteplase, and urokinase; direct thrombin inhibitors such as argatroban, bivalirudin, desirudin, lepirudin, melagatran, and ximelagatran; other antithrombotics such as dabigatran, defibrotide, dermatan sulfate, fondaparinux, and rivaroxaban; and others such as citrate, EDTA, and oxalate.

Sepsis and Disseminated Intravascular Coagulation

The methods and devices of the invention can be used to assess the hemostatic condition of subjects suffering from sepsis or disseminated intravascular coagulation.

In sepsis, an overwhelming inflammatory response causes extensive collateral damage to the host's microcirculation. Damage to the endothelium exposes tissue factor and in sepsis, which may occur on a large scale. Tissue factor, in turn, binds to activated factor VII. The resulting complex activates factors IX and X. Factor X converts prothrombin into thrombin, which cleaves fibrinogen into fibrin, inducing the formation of a blood clot. At the same time, the fibrinolytic system is inhibited. Cytokines and thrombin stimulate the release of plasminogen-activator inhibitor-1 (PAI-1) from platelets and the endothelium. When a clot forms in the human body, it is ultimately broken down by plasmin, which is activated by tissue plasminogen activator (TPA). PAI-1 inhibits TPA. Consequently, subjects suffering from severe sepsis are treated with an anticoagulant such as protein C (blood coagulant factor XIV).

Disseminated intravascular coagulation (DIC) is a complex systemic thrombohemorrhagic disorder involving the generation of intravascular fibrin and the consumption of procoagulants and platelets. The resultant clinical condition is characterized by intravascular coagulation and hemorrhage. DIC is not an illness on its own but rather a complication or an effect of progression of other illnesses and is estimated to be present in up to 1% of hospitalized patients. DIC is always secondary to an underlying disorder and is associated with a number of clinical conditions, generally involving activation of systemic inflammation. DIC has several consistent components including activation of intravascular coagulation, depletion of clotting factors, and end-organ damage. DIC is most commonly observed in severe sepsis and septic shock. Indeed, the development and severity of DIC correlates with mortality in severe sepsis. Although bacteremia, including both gram-positive and gram-negative organisms, is most commonly associated with DIC, other infections including viral, fungal, and parasitic infections may cause DIC. Trauma, especially neurotrauma, is also frequently associated with DIC. DIC is more frequently observed in those patients with trauma who develop the systemic inflammatory response syndrome. Evidence indicates that inflammatory cytokines play a central role in DIC in both trauma patients and septic patients. In fact, systemic cytokine profiles in both septic patients and trauma patients are nearly identical.

DIC exists in both acute and chronic forms. DIC develops acutely when sudden exposure of blood to procoagulants occurs, including tissue factor (tissue thromboplastin), generating intravascular coagulation. Compensatory hemostatic mechanisms are quickly overwhelmed, and, as a consequence, a severe consumptive coagulopathy leading to hemorrhage develops. Abnormalities of blood coagulation parameters are readily identified, and organ failure frequently occurs in acute DIC. In contrast, chronic DIC reflects a compensated state that develops when blood is continuously or intermittently exposed to small amounts of tissue factor. In chronic DIC, compensatory mechanisms in the liver and bone marrow are not overwhelmed, and there may be little obvious clinical or laboratory indication of the presence of DIC. Chronic DIC is more frequently observed in solid tumors and in large aortic aneurysms.

Exposure to tissue factor in the circulation occurs via endothelial disruption, tissue damage, or inflammatory or tumor cell expression of procoagulant molecules, including tissue factor. Tissue factor activates coagulation by the extrinsic pathway involving factor Vila. Factor Vila has been implicated as the central mediator of intravascular coagulation in sepsis. Blocking the factor Vila pathway in sepsis has been shown to prevent the development of DIC, whereas interrupting alternative pathways did not demonstrate any effect on clotting. The tissue factor-Vila complex then serves to activate thrombin, which, in turn, cleaves fibrinogen to fibrin while simultaneously causing platelet aggregation. Evidence suggests that the intrinsic (or contact) pathway is also activated in DIC, while contributing more to hemodynamic instability and hypotension than to activation of clotting.

Decreased levels of antithrombin correlate with elevated mortality in patients with sepsis. Thrombin generation is usually tightly regulated by multiple hemostatic mechanisms. Antithrombin function is one such mechanism responsible for regulating thrombin levels. However, due to multiple factors, antithrombin activity is reduced in patients with sepsis. First, antithrombin is continuously consumed by ongoing activation of coagulation. Moreover, elastase produced by activated neutrophils degrades antithrombin as well as other proteins. Further antithrombin is lost to capillary leakage. Lastly, production of antithrombin is impaired secondary to liver damage resulting from under-perfusion and microvascular coagulation.

Tissue factor pathway inhibitor (TFPI) depletion is evidence in subjects with DIC. TFPI inhibits the tissue factor-Vila complex. Although levels of TFPI are normal in patients with sepsis, a relative insufficiency in DIC is evident. TFPI depletion in animal models predisposes them to DIC, and TFPI blocks the procoagulant effect of endotoxin in humans. The intravascular fibrin produced by thrombin is normally eliminated via a process termed fibrinolysis. The initial response to inflammation appears to be augmentation of fibrinolytic action; however, this response soon reverses as inhibitors of fibrinolysis are released. High levels of PAI-1 precede DIC and predict poor clinical outcomes. Fibrinolysis cannot keep pace with increased fibrin formation, eventually resulting in under-opposed fibrin deposition in the vasculature.

Protein C, along with protein S, serves in important anticoagulant compensatory mechanisms. Under normal conditions, protein C is activated by thrombin and is complexed on the endothelial cell surface with thrombomodulin. Activated protein C combats coagulation via proteolytic cleavage of factors Va and VIIIa. However, cytokines (e.g., tumor necrosis factor α (TNF-α) and interleukin 1 (IL-1)) produced in sepsis and other generalized inflammatory states largely incapacitate the protein C pathway. Inflammatory cytokines down-regulate the expression of thrombomodulin on the endothelial cell surface. Protein C levels are further reduced via consumption, extravascular leakage, reduced hepatic production, and by a reduction in freely circulating protein S.

Inflammatory and coagulation pathways interact in substantial ways. Many of the activated coagulation factors produced in DIC contribute to the propagation of inflammation by stimulating endothelial cell release of proinflammatory cytokines. Factor Xa, thrombin, and the tissue factor-Vila complex have each been demonstrated to elicit proinflammatory action. Furthermore, given the anti-inflammatory action of activated protein C, its impairment in DIC contributes to further dysregulation of inflammation.

Components of DIC include: exposure of blood to procoagulant substances; fibrin deposition in the microvasculature; impaired fibrinolysis; depletion of coagulation factors and platelets (consumptive coagulopathy); organ damage and failure. DIC may occur in 30-50% of patients with sepsis.

The methods and devices of the invention may find use in monitoring subjects with a variety of DIC-associated conditions such as: sepsis/severe infection; trauma (neurotrauma); organ destruction; malignancy (solid and myeloproliferative malignancies); severe transfusion reactions; rheumatologic illness; obstetric complications (amniotic fluid embolism, abruptio placentae, hemolysis, retained dead fetus syndrome); vacular abnormalities (Kasabach-Merritt syndrome, aneurysms); hepatic failure; toxic reactions, transfusion reactions, and transplant rejections. Similarly, the invention may be used with respect to subjects having hemostatic conditions characterized by acute DIC associated with bacterial infections (e.g., gram-negative sepsis, gram-positive infections, or rickettsial), viral infections (e.g., associated with HIV, cytomegalovirus, varicella, or hepatitis), fungal infections, parasitic infection (e.g., malaria), malignancy (e.g., acute myelocytic leukemias), obstetric conditions (e.g., eclampsia placental abruption or amniotic fluid embolism), trauma, burns, transfusion, hemolytic reactions, or transplant rejection.

The NMR-based methods of the invention may be use to monitor any and all of the blood-related conditions described above. Time-domain relaxometry, particularly T2 relaxation measurements, can be used to measure a change in the clotting state of a sample. This measurement relies on measuring NMR parameters of the hydrogen nuclei that are sensitive to changes in the macroscopic clotting state of the sample. Most of the hydrogen nuclei are in the bulk water solvent, but an appreciable fraction of them are in the biological macromolecules and cells and platelets in the sample. As such, the measurement of the average NMR signal from all hydrogen nuclei can be conducted such that the signal changes in an appreciable manner when the clotting state of the sample changes for any of the clinical reasons described above. The NMR measurement can be a T2 relaxation measurement, or an “effective” T2 relaxation measurement (e.g., a T2 relaxation measurement where the parameters of the signal acquisition are such that they are set for optimal readout of the clotting event and not for the most accurate measurement of a T2 relaxation value). Other “time domain” relaxation measurement methods can be applied to measure changes in clotting behaviors. These may include time-domain free-induction decay analyses amongst other measurements. Any of the NMR time domain measurements described herein can be acquired in a repeated fashion to get a dynamic read-out of the NMR signal over the course of time as the clotting or dissolution properties of the sample change.

Subjects Having Normal and Abnormal Hemostatic Profiles

The methods of the invention can be used to discriminate between subjects having normal and abnormal hemostatic profiles. For example, the NMR relaxation parameter value and/or T2 signature characteristic of normal and abnormal hemostatic profiles can be determined and used in the differential diagnosis of a subject, such as a subject having sickle cell anemia. Abnormal hemostatic profiles can include profiles for subjects sharing a common deficiency in one or more clotting factors, clotting cofactors, and/or regulatory proteins (e.g., factor XII, factor XI, factor IX, factor VII, factor X, factor II, factor VIII, factor V, factor III (tissue factor), fibrinogen, factor I, factor XIII, von Willebrand factor, protein C, protein S, thrombomodulin, and antithrombin III, among others). The distinction of normal versus abnormal subjects can be indicative of disease states that are not from factor deficiencies.

A deficiency in antithrombin is seen in approximately 2% of patients with venous thromboembolic disease. Inheritance occurs as an autosomal dominant trait. The prevalence of symptomatic antithrombin deficiency ranges from 1 per 2000 to 1 per 5000 in the general population. Deficiencies results from mutations that affect synthesis or stability of antithrombin or from mutations that affect the protease and/or heparin binding sites of antithrombin. The methods of the invention can be used to discriminate between normal subjects and subjects having a deficiency in antithrombin.

A deficiency in factor XI confers an injury-related bleeding tendency. This deficiency was identified in 1953 and originally termed hemophilia C. Factor XI deficiency is very common in Ashkenazic Jews and is inherited as an autosomal disorder with either homozygosity or compound heterozygosity. The methods of the invention can be used to discriminate between normal subjects and subjects having a deficiency in factor XI.

von Willebrand disease (vWD) is due to inherited deficiency in von Willebrand factor (vWF). vWD is the most common inherited bleeding disorder of humans. Deficiency of vWF results in defective platelet adhesion and causes a secondary deficiency in factor VIII. The result is that vWF deficiency can cause bleeding that appears similar to that caused by platelet dysfunction or hemophilia. vWD is an extremely heterogeneous disorder that has been classified into several major subtypes. Type I vWD is the most common and is inherited as an autosomal dominant trait. This variant is due to simple quantitative deficiency of all vWF multimers. Type 2 vWD is also subdivided further dependent upon whether the dysfunctional protein has decreased or paradoxically increased function in certain laboratory tests of binding to platelets. Type 3 vWD is clinically severe and is characterized by recessive inheritance and virtual absence of vWF. The methods of the invention can be used to discriminate between normal subjects and subjects having a deficiency in von Willebrand factor.

Several cardiovascular risk factors are associated with abnormalities in fibrinogen. Elevated plasma fibrinogen levels have been observed in patients with coronary artery disease, diabetes, hypertension, peripheral artery disease, hyperlipoproteinemia and hypertriglyceridemia. In addition, pregnancy, menopause, hypercholesterolemia, use of oral contraceptives and smoking lead to increased plasma fibrinogen levels. There are inherited disorders in fibrinogen, including afibrinogenemia (a complete lack of fibrinogen), hypofibrinogenemia (reduced levels of fibrinogen) and dysfibrinogenemia (presence of dysfunctional fibrinogen). Afibrinogenemia is characterized by neonatal umbilical cord hemorrhage, ecchymoses, mucosal hemorrhage, internal hemorrhage, and recurrent abortion. The disorder is inherited in an autosomal recessive manner. Hypofibrinogenemia is characterized by fibrinogen levels below 100 mg/dL (normal is 250-350 mg/dL) and can be either acquired or inherited. The methods of the invention can be used to discriminate between normal subjects and subjects having abnormalities in fibrinogen.

Platelet Monitoring

The methods and device of the invention can be used to determine platelet function and be compared to platelet aggregometry (see, e.g., Harris et al., Thrombosis Research 120:323 (2007)). Currently there are two detection methods used in instruments with FDA clearance for performing platelet aggregometry: optical and impedance measurements. For example, the methods of the invention can be used to identify any platelet activity or diagnose any platelet dysfunction in a subject that may be measured by platelet aggregometry. Platelet aggregometry is a functional test performed on a whole blood or platelet-rich plasma sample. Generally, platelet aggregometry methods involve adding a platelet activator to the sample and measuring the induced platelet aggregation. Platelet aggregometry can be performed by immersing an electrode in the blood sample being tested. Platelets adhering to the probe form a stable monolayer. When an activator is added, platelet aggregates form on the electrode and increase the resistance to a current being applied across the electrode. The instrument monitors the change in electrical impedance, which reflects the platelet aggregation response. Aggregometry methods also include techniques based on monitoring the release of ATP from aggregating platelets by luminescence. Optical detection of platelet aggregation is based on the observation that, as platelets aggregate into large clumps, there is an increase in light transmittance. Different aggregation-inducing agents stimulate different pathways of activation and different patterns of aggregation are observed. The main drawback of the optical method is that it is typically performed on PRP, necessitating the separation of platelets from red blood cells and adjustment of the platelet count to a standardized value.

As in platelet aggregometry, the methods of the invention may be used assess the platelet count from a blood sample of a subject or to diagnose a condition of thrombocytopenia (platelet count<150,000/μL) or thrombocytosis (platelet count>400,000/μL) in a subject. Such a diagnosis may be used as the basis of a decision to provide the subject with a platelet transfusion or an anticoagulant. Similarly, the methods of the invention may be used to evaluate the response of a subject to a platelet transfusion or an anticoagulant.

T2MR Coagulopathy Panel

Unlike other hemostasis measurement tools, T2MR enables multiplexed hemostasis measurements for different hemostasis parameters that are normally analyzed as single assays on separate and distinct platforms. These multiplexed measurements enable novel combination of diagnostic assays that may not been possible on a single instrument or assay panel, especially at the point of care. The T2MR based assay panel can be conveniently used at point of care and in a hospital laboratory.

A specific T2MR assay panel has been designed to aid in the diagnosis and treatment of patients suffering from trauma, undergoing treatment in the operating room, or who have an underlying complicated disease or disorder that requires a multifaceted diagnostic analysis. The information provided by these assays aids in the appropriate decisions for transfusion, administration of therapeutics to restore hemostasis, and other medical interventions.

The T2MR coagulopathy panel allows for measurement of multiple categories of hemostasis parameters in whole blood, eliminating the need for time-consuming sample preparation. These include (1) clotting time parameters, (2) hematocrit or hemoglobin levels, (3) global platelet activity/inhibition measurements, (4) fibrinogen measurements, and (5) fibrinolysis measurements. These parameters are multiplexed in the diagnostic panel. Multiplexing can take place as either (1) a single-reaction multiplexed result, (2) measurements obtained in parallel from multiple aliquots of the same sample, or (3) measurements obtained in succession on the same instrument with multiple aliquots of the sample.

The T2MR coagulopathy panel measures multiple clinical parameters that are important for the management of patients that have experienced or are suspected of trauma, including surgical trauma. Assessing these parameters for patients who have experienced or are suspected of trauma is valuable for two reasons: 1) diagnosing acute coagulopathy, which is often caused by trauma, and 2) directing appropriate therapy for patients that are in need of transfusion products. Trauma may occur in many settings: 1) accidents—auto and otherwise, 2) combat, 3) results of violent acts, including gunshots, 4) birth, 5) sporting events, 6) surgery, and any event that may lead to blunt or penetrating wounds. Given the importance of identifying trauma early to assist with improved outcomes, identifying these factors is valuable at the point of trauma (battlefield, site of injury—sporting event, auto accident, point of gunshot wound, operating room), along the path towards treatment (medivac, helicopter, ambulance), at the point of hospital admission (trauma triage center, emergency room), or in a centralized setting (hospital laboratory). There are many causes and mechanisms leading to coagulopathy as a result of trauma (see Hess et al., J. Trauma 65:748 (2008), incorporated herein by reference). For example, there are known clinical syndromes occurring after trauma: dilutional coagulopathy, the fatal triad of shock, acidosis and hypothermia, and acute coagulopathy of trauma-shock or ACoTS. While trauma leads to a majority of coagulopathies, it is also known that coagulopathy can be associated with other disease/disorders, medications, and genetic predispositions.

This T2MR comprehensive panel can identify an acute coagulopathy, which will provide the information necessary to prompt an intervention, while the specific data will also direct the appropriate transfusion product. For instance, low hematocrit may lead toward red blood cell replacement, low fibrinogen may lead to fibrinogen treatment or fresh frozen plasma (FFP) administration, abnormal clot time may lead to administration of clotting factors or FFP, and abnormal platelet activity will suggest a platelet transfusion or appropriate medication. Because the effect of trauma on the coagulation state on a specific patient is unknown, each measured parameter may be used individually or in combination with all others. Based on the results of factor deficiency, abnormal activity, or other abnormalities in results, the specific therapy may be chosen. A broad assessment of clotting time, hematocrit levels, fibrinogen levels and platelet activity, along with other factors will provide the most appropriate transfusion decisions or therapeutic actions, and algorithms will vary based on clinical evidence; however, a potential approach is described in Maegle et al., World J. Emerg. Med. 1:12 (2010).

The T2MR primary assay configuration that enables a simple multiplexed coagulopathy panel is an assay configuration where a single activator is used to trigger clotting in whole blood. This activator not only triggers enzymatic coagulation and subsequent fibrin formation but also triggers platelet activity and subsequent clot contraction. The former allows for measurement of clotting time and defects or inhibition in the enzymatic cascade and the latter allows for measurement of platelet activity or inhibition as well as abnormally low fibrinogen levels. This multiplex capability distinguishes T2MR technology from thromboelastography, which is unable to measure PT-like clotting times and has been show to be insensitive to measurement of fibrinolysis and is unable to easily measure and distinguish fibrin contribution to clot strength from platelet contribution to clot strength. Additionally, measurement of the T2MR signals during the initial portion of the reaction enable determination of hematocrit. Lastly, if desired, the measurements can be used to monitor deficiencies in fibrinolysis by monitoring signals after clot contraction or monitoring deficiencies in clot formation or contraction and in this case an additional reagent may be required in the assay, such as aprotinin to inhibit fibrinolysis and compare signature to those obtained in the absence of aprotinin. In essence, this clotting activator cocktail enables measurement of global hemostasis performance including hematocrit, enzymatic cascade, platelets, and fibrinogen.

The activation cocktail is an important feature of the T2MR coagulopathy panel. The activation cocktail may be composed of one or more activators, initiators, or compounds required for the reaction to occur. In one embodiment it consists of a diluted PT reaction, which can be initiated with Innovin at a specific concentration. Innovin can be replaced more generally with any preparation of ‘tissue factor’ and lipid′; tissue factor to include both recombinant and non-recombinant, and lipid to include defined and undefined mixtures of phospholipids as well as Cephalin or any other natural substitute for platelet phospholipid. In another embodiment of the invention the activation cocktail initiates an EXTEM reaction. In this embodiment, the standard ratio of EXTEM to citrated whole blood can be used at volumes up to and including our sampling limit (typically 40-60 μL); for example, 2.4 μL EXTEM reagent plus 35.3 μL citrated whole blood plus 2.4 μL STAR-TEM produces an activation signal within 10 minutes of the start of the reaction. In still another embodiment the activation cocktail initiates a kaolin activation. In this example, 34 μL of citrated whole blood can be mixed with 1 μL of the kaolin solution (Haemonetics) and 2 μL of 0.2M CaCl₂ solution. Normal sample activation is observed in 4 to 8 minutes). In this assay format, the kaolin+/−calcium solution may be in a dried form in the reactant tube.

In other embodiments, the activator can be tissue factor (recombinant human tissue factor), contact factor/aPTT reagent (such as celite, ellagic acid, or kaolin), tissue factor or contact factor activator plus cytochalasin D or Reopro (which blocks platelet activation), tissue factor+aprotinin (which blocks fibrinolysis), phospholipid, celite, or thrombin, among others. All of these activators can be combined with calcium for use with citrated blood. With this set of tests, the main pathways of clot formation and fibrinolysis can be measured. These activators can be combined with levels of protamine or heparinase as an aid in identifying heparin-mediated affects, or with ADP, arachidonic acid, serotonin, epinephrine, ristocetin, collagen, as well as the application of heat, cold, or vigorous mixing to cause platelet activation. Protamine will reverse the effects of heparin by binding quantitatively to it; the addition of 1 mg/ml protamine sulfate for each 100 IU/ml heparin will reverse the anticoagulant activity of heparin. A cocktail that contains protamine sulfate is expected to reverse the effects of heparin anticoagulation but have little effect on other mechanisms that inhibit clotting, such as factor deficiencies.

The activator selection is critical so that the desired sensitivities are achieved. For example, the activator will determine whether the clotting phenomenon being measured is a fast clotting test (like PT) or moderate (like PTT, or ACT) or slow clotting tests (like R—as in TEG parameter R). Additionally, the selection of the activator will determine which anti-coagulants the clotting time measurement will be sensitive to. These may include different combinations of warfarin, rivaroxaban, dabigatran, heparin, hirudin, or direct thrombin inhibitors, among others. The multiplex coagulopathy panel can be tailored to different patient states; for example, a cardiac bypass patient on unfractionated heparin (UFH) might require a cocktail with low-level protamine, whereas a patient infused with blood diluents may require a higher level of tissue factor to adequately monitor the state of the patient during surgery. The method can accommodate a variety of cocktail mixtures.

Optionally, the coagulopathy panel assay is carried out using a disposable reaction tube or cartridge that is loaded with reaction activators and sample using a pipette. In another embodiment the disposable has been pre-loaded with a dried or frozen activator cocktail and the sample is loaded with a pipette. In still another embodiment the disposable allows addition of blood in a non quantified manner and the disposable combines the blood with the reactions in a controlled fashion. The disposable may, for example, have the ability to split the sample between different reaction tubes to permit either simultaneous or concurrent tests to be performed on the same sample of blood.

Sample Tubes

The methods of the invention include measures for evaluating hemostatic conditions and parameters through the observation of platelet-induced clot contraction. These include platelet activity, hyper and hypocoagulability states, and clot lysis, among others. The kinetics and signals associated with these reactions depend on at least three categories of variables: (1) the inherent biology within the sample, such as platelet activity, factor deficiencies, and therapeutic agents; (2) the type and concentration of specific activator used to initiate clotting in the sample; and (3) variation in how the clot forms and contracts within the sample tube. One goal of the methods of the invention is to ensure that the variability in the observed experimental values reflects only variability in the inherent biology of the sample (category 1). To this end, standard reagent formulations can be used to control and reduce variability arising from the predetermined condition of clot initiation (category 2) for any given sample measurement. We have observed that variability in fibrin adhesion to the inner surface of the sample tube (category 3) can sometimes introduce variability in the sample measures that can reduce the sensitivity and reproducibility of the methods of the invention. To reduce this source of variability the methods of the invention can be performed in a sample tube having an inner surface that controls fibrin adhesion. The use of sample tubes that control fibrin adhesion can result in more robust, sensitive, and reproducible clot-contraction based assays, thereby producing more accurate data that correlates better with reference methods and clinical outcomes.

The sample tubes used in the methods of the invention can include an inner surface of the sample tube that controls fibrin adhesion. This can be achieved through the selection of an appropriate material from which the entire sample tube is made, or by coating the inner surface of a sample tube (covalently or non-covalently) with a material that controls fibrin adhesion. For example, the inner surface can include a fluorinated material or a pegylated material or a material that increases the hydrophilicity of the inner surface to impart resistance to fibrin adhesion. The inner surface can include a substrate coated with a material that reduces fibrin adhesion in comparison to the substrate uncoated. The substrate can be, for example, glass or a base polymer (e.g., polypropylene, polycarbonate, polystyrene, polyallomer, or another base polymer suitable for making into a sample tube). The substrate can be a glass coated by silanization with a material that reduces fibrin adhesion in comparison to unsilanized glass. Alternatively, the material includes a surfactant, a polynucleotide, a protein, a polyethyle glycol, a fluorinated material (e.g., fluorocarbon coating), hydrophilic polymers (e.g. polyacrylates, polyvinyl alcohol, etc.), a carbohydrate (e.g., agarose, cellulose, carboxymethyl cellulose), or a mixture thereof.

The sample tubes used in the methods of the invention can include an inner surface conditioned/processed (e.g., silanization, siliconization, thin film deposition, plasma etching, plasma cleaning, etc.) to resist fibrin adhesion. Such processing can include plasma cleaning (i.e., corona treatment) to remove contaminants from the inner surface of the tube, or to prepare the surface for coating with a material that resists fibrin adhesion, or to produce a smoother substrate surface that controls fibrin adhesion. For example, the sample tubes used in the methods of the invention can include an inner surface patterned with hydrophilic and hydrophobic groups on the underlying substrate of the sample to tube, a feature reported to reduce fibrin adhesion in contact lenses (see Sato et al., Proc. SPIE 5688, Ophthalmic Technologies XV, 260 (2005)). Alternatively, the sample tubes can include a thin film deposited onto the surface, such as a thin film including polyethylene glycol, fluorinated material, or a noble metal (e.g., silver, gold, platinum, palladium). In still another approach, the inner surface of the sample tube can be subjected to chemical vapor deposited poly(p-xylylene) polymers (i.e., a parylene coating).

The sample tubes used in the methods of the invention can include an inner surface bearing one or more materials having an extremely low coefficient of friction to provide a non-stick surface, such as polytetrafluoroethylene (Teflon®), fluorinated ethylene-propylene (FEP), perfluoroalkoxy polymer resin (PFA), parafilm (i.e., a surface coated with paraffin wax), or silicone. The sample tubes used in the methods of the invention can include an inner surface formed from a base polymer free of additives (e.g., lubricants, plasticizers, colorants, and other commonly used additives) which can migrate to the surface of the base polymer and alter its surface properties. For example, the inner surface can be formed from high purity polystyrene (e.g., Dow 666U), or a high purity polyacrylic acid (e.g., PMMA). The base polymer optionally can be selected to provide a hydrophilic inner surface, or is covalently modified (e.g., by oxygen plasma coating, air plasma coating, UV activated coating, or direct oxidation, e.g., with permanganate, to produce surface carboxylate groups) to provide a hydrophilic inner surface. The hydrophilic inner surface can be produced by controlling the presence of electronegative functional groups, such as functional groups containing nitrogen and/or oxygen.

The sample tubes used in the methods of the invention can include an inner surface including a substrate (e.g., glass or a base polymer, such as polypropylene, polycarbonate, polystyrene, polyallomer, or another base polymer suitable for making into a sample tube) coated with a surfactant. The surfactant may be selected from a wide variety of soluble non-ionic surface active agents including surfactants that are generally commercially available under the IGEPAL trade name from GAF Company. The IGEPAL liquid non-ionic surfactants are polyethylene glycol p-isooctylphenyl ether compounds and are available in various molecular weight designations, for example, IGEPAL CA720, IGEPAL CA630, and IGEPAL CA890. Other suitable non-ionic surfactants include those available under the trade name TETRONIC 909 from BASF Wyandotte Corporation. This material is a tetra-functional block copolymer surfactant terminating in primary hydroxyl groups. Suitable non-ionic surfactants are also available under the VISTA ALPHONIC trade name from Vista Chemical Company and such materials are ethoxylates that are non-ionic biodegradables derived from linear primary alcohol blends of various molecular weights. The surfactant may also be selected from poloxamers, such as polyoxyethylene-polyoxypropylene block copolymers, such as those available under the trade names Synperonic PE series (ICI), Pluronic® series (BASF), Supronic, Monolan, Pluracare, and Plurodac; polysorbate surfactants, such as Tween® 20 (PEG-20 sorbitan monolaurate); nonionic detergents (e.g., nonyl phenoxypolyethoxylethanol (NP-40), 4-octylphenol polyethoxylate (Triton-X100), Brij nonionic surfactants); and glycols such as ethylene glycol and propylene glycol.

The surfactant can be, for example, a polyethylene glycol alkyl ether or polysorbate surfactant.

Polyethylene glycol alkyl ether surfactants can be used to coat the sample tubes utilized in the methods of the invention, and include, without limitation, Laureth 9, Laureth 12 and Laureth 20. Other polyethylene glycol alkyl ethers include, without limitation, PEG-2 oleyl ether, oleth-2 (Brij 92/93, Atlas/ICI); PEG-3 oleyl ether, oleth-3 (Volpo 3, Croda); PEG-5 oleyl ether, oleth-5 (Volpo 5, Croda); PEG-10 oleyl ether, oleth-10 (Volpo 10, Croda, Brij 96/97 12, Atlas/ICI); PEG-20 oleyl ether, oleth-20 (Volpo 20, Croda, Brij 98/99 15, Atlas/ICI); PEG-4 lauryl ether, laureth-4 (Brij 30, Atlas/ICI); PEG-9 lauryl ether; PEG-23 lauryl ether, laureth-23 (Brij 35, Atlas/ICI); PEG-2 cetyl ether (Brij 52, ICI); PEG-10 cetyl ether (Brij 56, ICI); PEG-20 cetyl ether (Brij 58, ICI); PEG-2 stearyl ether (Brij 72, ICI); PEG-10 stearyl ether (Brij 76, ICI); PEG-20 stearyl ether (Brij 78, ICI); and PEG-100 stearyl ether (Brij 700, ICI). Polysorbate surfactants can be used to coat the sample tubes utilized in the methods of the invention. Polysorbate surfactants are oily liquids derived from pegylated sorbitan esterified with fatty acids. Common brand names for Polysorbates include Alkest, Canarcel and Tween. Polysorbate surfactants include, without limitation, polyoxyethylene 20 sorbitan monolaurate (TWEEN 20), polyoxyethylene (4) sorbitan monolaurate (TWEEN 21), polyoxyethylene 20 sorbitan monopalmitate (TWEEN 40), polyoxyethylene 20 sorbitan monostearate (TWEEN 60); and polyoxyethylene 20 sorbitan monooleate (TWEEN 80).

In some cases, an RF coil maybe integrated into a disposable sample tube and be a disposable component of the system used to perform the methods of the invention. The coil may be placed in a manner that allows electrical contact with circuitry on the fixed NMR setup, or the coupling may be made inductively to a circuit.

T2MR Units

The systems for carrying out the methods of the invention can include one or more NMR units. A bias magnet establishes a bias magnetic field B₀ through a sample. An RF coil and RF oscillator provides an RF excitation at the Larmor frequency which is a linear function of the bias magnetic field B₀. In one embodiment, the RF coil is wrapped around the sample well. The excitation RF creates a nonequilibrium distribution in the spin of the water protons (or free protons in a non-aqueous solvent). When the RF excitation is turned off, the protons “relax” to their original state and emit an RF signal that can be used to extract information about the water populations in the blood sample. The coil acts as an RF antenna and detects a signal, which based on the applied RF pulse sequence, probes different properties of the material, for example a T₂ relaxation. The signal of interest for some cases of the technology is the spin-spin relaxation (generally 10-2000 milliseconds) and is called the T₂ relaxation. The RF signal from the coil is amplified and processed to determine the T₂ (decay time) response to the excitation in the bias field B₀. The well may be a small capillary or other tube with nanoliters to microliters of the sample, including the blood sample and an appropriately sized coil wound around it. The coil is typically wrapped around the sample and sized according to the sample volume. For example (and without limitation), for a sample having a volume of about 10 ml, a solenoid coil about 50 mm in length and 10 to 20 mm in diameter could be used; for a sample having a volume of about 40 μL, a solenoid coil about 6 to 7 mm in length and 3.5 to 4 mm in diameter could be used; and for a sample having a volume of about 0.1 nl a solenoid coil about 20 μm in length and about 10 μm in diameter could be used. Alternatively, the coil may be configured within, about, or in proximity to the well or sample tube. An NMR system may also contain multiple RF coils for the detection of multiplexing purposes. In certain embodiments, the RF coil has a conical shape with the dimensions 6 mm×6 mm×2 mm.

The NMR unit includes a magnet (i.e., a superconducting magnet, an electromagnet, or a permanent magnet). The magnet design can be open or partially closed, ranging from U- or C-shaped magnets, to magnets with three and four posts, to fully enclosed magnets with small openings for sample placement. The tradeoff is accessibility to the “sweet spot” of the magnet and mechanical stability (mechanical stability can be an issue where high field homogeneity is desired). For example, the NMR unit can include one or more permanent magnets, cylindrically shaped and made from SmCo, NdFeB, or other low field permanent magnets that provide a magnetic field in the range of about 0.5 to about 1.5 T (i.e., suitable SmCo and NdFeB permanent magnets are available from Neomax, Osaka, Japan). For purposes of illustration and not limitation, such permanent magnets can be a dipole/box permanent magnet (PM) assembly, or a hallbach design (See Demas et al., Concepts Magn Reson Part A 34A:48 (2009)). The NMR units can include, without limitation, a permanent magnet of about 0.5 T strength with a field homogeneity of about 20-30 ppm and a sweet spot of 40 μL, centered. This field homogeneity allows a less expensive magnet to be used (less tine fine-tuning the assembly/shimming), in a system less prone to fluctuations (e.g. temperature drift, mechanical stability over time-practically any impact is much too small to be seen), tolerating movement of ferromagnetic or conducting objects in the stray field (these have less of an impact, hence less shielding is needed), without compromising the assay measurements (relaxation measurements and correlation measurements do not require a highly homogeneous field).

The basic components of an NMR unit include electrical components, such as a tuned RF circuit within a magnetic field, including an MR sensor, receiver and transmitter electronics that could be including preamplifiers, amplifiers and protection circuits, data acquisitions components, pulse programmer and pulse generator.

The NMR system may include a chip with RF coil(s) and electronics micro-machined thereon. For example, the chip may be surface micromachined, such that structures are built on top of a substrate. Where the structures are built on top of the substrate and not inside it, the properties of the substrate are not as important as in bulk micromachining, and expensive silicon wafers used in bulk micromachining can be replaced by less expensive materials such as glass or plastic. Alternative embodiments, however, may include chips that are bulk micro-machined. Surface micromachining generally starts with a wafer or other substrate and grows layers on top. These layers are selectively etched by photolithography and either a wet etch involving an acid or a dry etch involving an ionized gas, or plasma. Dry etching can combine chemical etching with physical etching, or ion bombardment of the material. Surface micromachining may involve as many layers as is needed.

In some cases, an inexpensive RF coil maybe integrated into a disposable sample tube of the invention, or into a disposable cartridge. The coil could be placed in a manner that allows electrical contact with circuitry on the fixed NMR setup, or the coupling could be made inductively to a circuit.

Where the relaxation measurement is T₂, accuracy and repeatability (precision) will be a function of temperature stability of the sample as relevant to the calibration, the stability of the assay, the signal-to-noise ratio (S/N), the pulse sequence for refocusing (e.g., CPMG, BIRD, Tango, and the like), as well as signal processing factors, such as signal conditioning (e.g., amplification, rectification, and/or digitization of the echo signals), time/frequency domain transformation, and signal processing algorithms used. Signal-to-noise ratio is a function of the magnetic bias field (B₀), sample volume, filling factor, coil geometry, coil Q-factor, electronics bandwidth, amplifier noise, and temperature.

In order to understand the required precision of the T₂ measurement, one should look at a response curve of the assay at hand and correlate the desired precision of determining the water populations present in the blood sample and the precision of the measureable, e.g., T₂ for some cases. Then a proper error budget can be formed. The NMR units for use in the systems and methods of the invention can be those described in U.S. Pat. No. 7,564,245, incorporated herein by reference. The NMR units of the invention can include a small probehead for use in a portable magnetic resonance relaxometer as described in PCT Publication No. WO09/061481, incorporated herein by reference.

The systems of the invention can include a disposable sample tube or sample holder for use with the MR reader that is configured to permit a predetermined number of measurements (i.e., is designed for a limited number of uses). The disposable sample tube or sample holder can include none, part, or all, of the elements of the RF detection coil (i.e., such that the MR reader lacks a detection coil). For example, the disposable sample tube or sample holder can include a “read” coil for RF detection that is inductively coupled to a “pickup” coil present in the MR reader. When the sample container is inside the MR reader it is in close proximity to the pickup coil and can be used to measure NMR signal. Alternatively, the disposable sample tube or sample holder includes an RF coil for RF detection that is electrically connected to the MR reader upon insertion of the sample container. Thus, when the sample container is inserted into the MR reader the appropriate electrical connection is established to allow for detection. The number of uses available to each disposable sample tube or sample holder can be controlled by disabling a fusible link included either in the electrical circuit within the disposable sample holder, or between the disposable sample tube or sample holder and the MR reader. After the disposable sample tube or sample holder is used to detect an NMR relaxation in a sample, the instrument can be configure to apply excess current to the fusible link, causing the link to break and rendering the coil inoperable. Optionally, multiple fusible links could be used, working in parallel, each connecting to a pickup on the system, and each broken individually at each use until all are broken and the disposable sample tube or sample holder rendered inoperable. Preferably, the disposable sample tube is a coated tube of the invention.

### Examples

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

We demonstrate that the transverse relaxation time of the nuclear magnetic resonance signal of water, referred to here as T2MR, can be utilized to probe microenvironments of water molecules in blood ex vivo formed during hemostatic processes in a reagent-free manner. Our results show that T2MR allows the physical states of blood to be monitored by continuously measuring the spin-spin (T2) relaxation times of water in a whole blood sample. Water is a sensitive and general magnetic resonance probe of the diverse and distinct microenvironments that develop during clot formation and structural rearrangement. For example, addition of an activator such as thrombin to whole blood initiates platelet aggregation and fibrin polymerization, generating a clot that subsequently undergoes platelet-mediated contraction. Contraction of the fibrin clot impacts microenvironments of water around the various components within the blood sample, including soluble proteins, erythrocytes, and the fibrin network itself, leading to the formation of multiple water compartments. These compartments and their formation over time can be discerned by applying an algorithm to resolve multiple time constants from a single T2MR relaxation curve. The sensitivity of the T2MR diagnostic platform to the hemostatic potential of blood arises from measuring these heterogeneities in the microenvironments of multiple water compartments that develop during clotting, contraction and lysis.

Here we describe how T2MR reports on the integrated contributions of plasma, platelets and other blood cells to hemostasis. This mix-and-read platform requires minimal sample volumes (less than 50 μl) compared with conventional methods and enables the measurement of both established and newly described hemostatic parameters on a single, simple to use instrument using water to probe the coagulative behavior of blood. This methodology can be used to measure both individual hemostatic parameters and integrated hemostasis. Major advantages over existing methods for measuring standard parameters include ease of performance by eliminating sample modification prior to analysis, data output in as little as a few minutes with the option to monitor samples for hours, and volume requirements that are 10-100 times less than existing methodologies.

Magnetic Resonance Relaxation Data

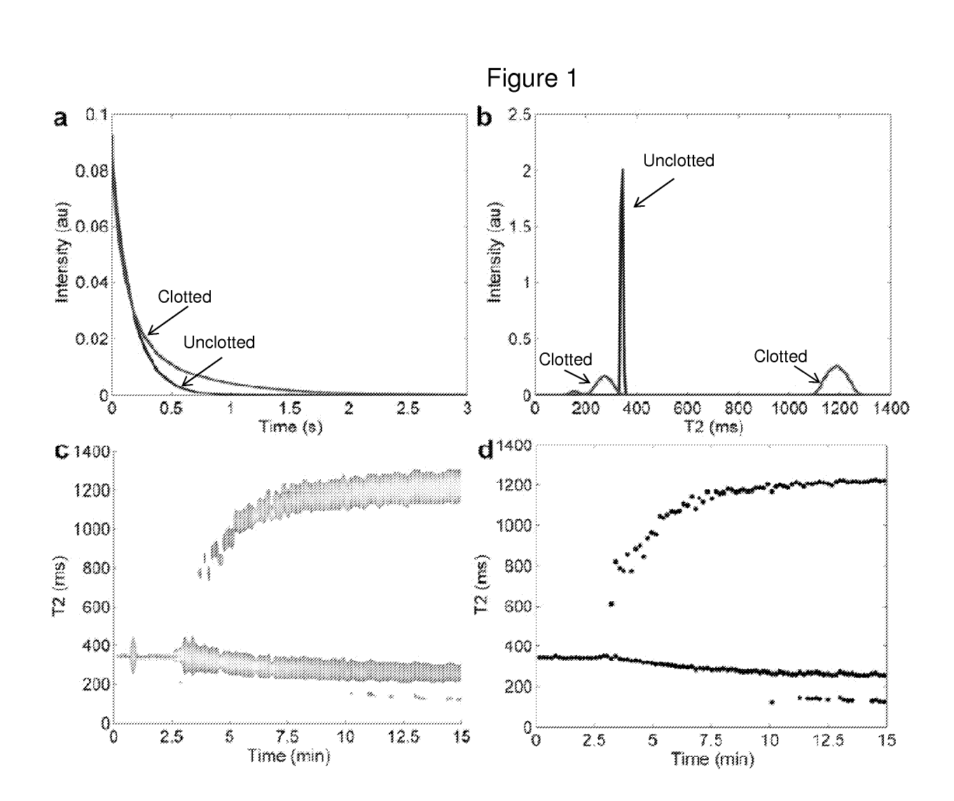

The relaxation mechanisms for magnetic resonance measurements of aqueous samples depend on chemical and diffusive exchange of water. A single relaxation value is measured when exchange is rapid, but multiple relaxation values can be measured when there is a barrier to exchange between microscopic environments. Key to applying T2MR to monitor microenvironment changes is the ability to resolve specific T2 relaxation values of multiple water compartments within a sample. This is achieved by implementing an algorithm based on the inverse Laplace transform, which has been applied previously to estimate component decay constants in exponential decay curves. Inverse Laplace transform processing of CPMG spectra produces a multi-exponential fit of the relaxation data shown in equation 9:

$$S ( t ) = {\sum }_{i}^{ } {A}_{i} {e}^{- t / {T2}_{i}} + O \qquad ( 9 )$$

where S(t) is the relaxation signal acquired with the CPMG sequence, A, is the amplitude corresponding to the relaxation time constant, T2ᵢ, and O is the offset term. FIGS. 1a-1d show how kinetic spectra can be formed from numerical inverse Laplace transform.

The precision and reproducibility of multi-component relaxation measurements across three T2MR instruments was characterized using mineral oil, which generates a two-component signal. Average T2 relaxation times (30 min measurements at sampling rate of 10 s) were 278 ms and 116 ms; average within-run precision (coefficient of variation (% CV)) values were 2.94% and 5.07% for the higher and lower component, respectively; day-to-day reproducibility (34 runs spanning 6 months) values were 3.4% and 7.6% for the higher and lower component, respectively.

Blood Sample Collection and Fractionation

Blood was obtained from healthy volunteers not taking aspirin, non-steroidal anti-inflammatory drugs or other medications known to inhibit platelet function for least 7-10 days, with informed consent and approval by Perelman School of Medicine-University of Pennsylvania Institutional Review Board. Blood was drawn via venipuncture into 3.2% trisodium citrate (9:1) following standard procedures that minimize platelet activation. Samples were kept at room temperature and were studied within 4 hr after the blood draw. A complete blood count was performed on an automated hematology analyzer (HemaVet 950FS, Drew Scientific, Dallas, Tex.).

For embodiments requiring fractionation and reconstitution of samples, 12 ml of blood was placed in 15 ml polypropylene tubes (Corning, Tewksbury, Mass.) and centrifuged for 15 min at 210 g at ambient temperature (22° C.). Platelet-rich plasma (PRP) was recovered from upper layer in the tube following centrifugation and transferred to a new tube. The residual blood preparation was centrifuged again at 900 g for 10 min at ambient temperature. The platelet-poor plasma (PPP) fraction was collected from the top layer and transferred to a new tube. Any remaining volume of PPP along with the buffy coat layer was removed, and the upper 1 ml of packed erythrocytes was aspirated and transferred to a new tube. To obtain concentrated platelets, PRP was centrifuged at 900 g for 10 min at ambient temperature. To prevent platelet aggregation, prostaglandin E1 (PGE1) was added (final concentration 5 μM). The supernatant was aspirated and discarded, and the platelet pellet was resuspended in PPP not containing PGE1 to generate a concentrated platelet suspension. Reconstituted samples were prepared by mixing concentrated erythrocytes, concentrated platelets, and PPP at desired levels.

Instrument Fabrication and Pulse Sequence Parameters

A small, portable T2MR instrument (35×15×18 cm, 9 kg) was designed to measure the proton T2 relaxation times within blood samples. The instrument consists of a 0.54 T (approximately 23 MHz) permanent magnet assembly, radiofrequency probe, single-board spectrometer, and peripheral electronics within a 37° C. temperature controlled enclosure. The radiofrequency probe accommodates 10-40 μL samples contained within a standard 0.2 ml polypropylene tube. A Carr-Purcell-Meiboom-Gill (CPMG) pulse sequence is applied to generate relaxation curves from which T2 values are extracted. The parameters of pulse sequence experiments were: inter-echo spacing (t_{E})=500 μs and repetition time (TR)=2−10 s depending on the application. This acquisition method removes the effects of static field inhomogeneities, enabling the use of a small, inexpensive magnet which is shimmed only once during manufacturing.

### Example 1

### Blood Clotting, Retraction, and Lysis with Thrombin and Tissue Plasminogen Activator

Blood clotting was initiated by addition of 2 μL of a 0.2 M CaCl₂ solution and 2 μL of thrombin (Sigma-Aldrich, St. Louis, Mo., final concentration 0.1-3.0 U/ml) to 34 μL of blood in a 200 μL PCR tube (Eppendorf, Hauppauge, N.Y.). All components were pre-warmed for 1 min at 37° C. before mixing. Samples were mixed by three aspiration and dispersion cycles using a pipette, then put into the T2MR reader for measurement. Typical run length was 30 min with a 10 s sampling rate. For some experiments, data collection time was extended to 1 hr.

To establish T2MR signatures for fibrinolysis, tissue plasminogen activator (tPA, Alteplase, Genentech, South San Francisco, Calif.) was added to samples clotted by thrombin. Blood clotting was initiated as described above, and the sample was incubated for 60 min to allow for complete clot contraction. Then, 0.5-1 μM tPA was added to clotted and contracted samples. Care was taken not to disturb clots, which were usually attached to the tube wall. The pipette tip was carefully placed into the visible serum layer on the tube side opposite to the contracted clot, and 3 μL of tPA solution was added with a single dispensing of the pipettor. The tPA solutions were made from a stock solution prepared according to manufacturer instructions using 0.15 M sodium chloride, pH 7.4.

### Example 2

### Real-Time Monitoring of Clot Formation, Contraction and Fibrinolysis

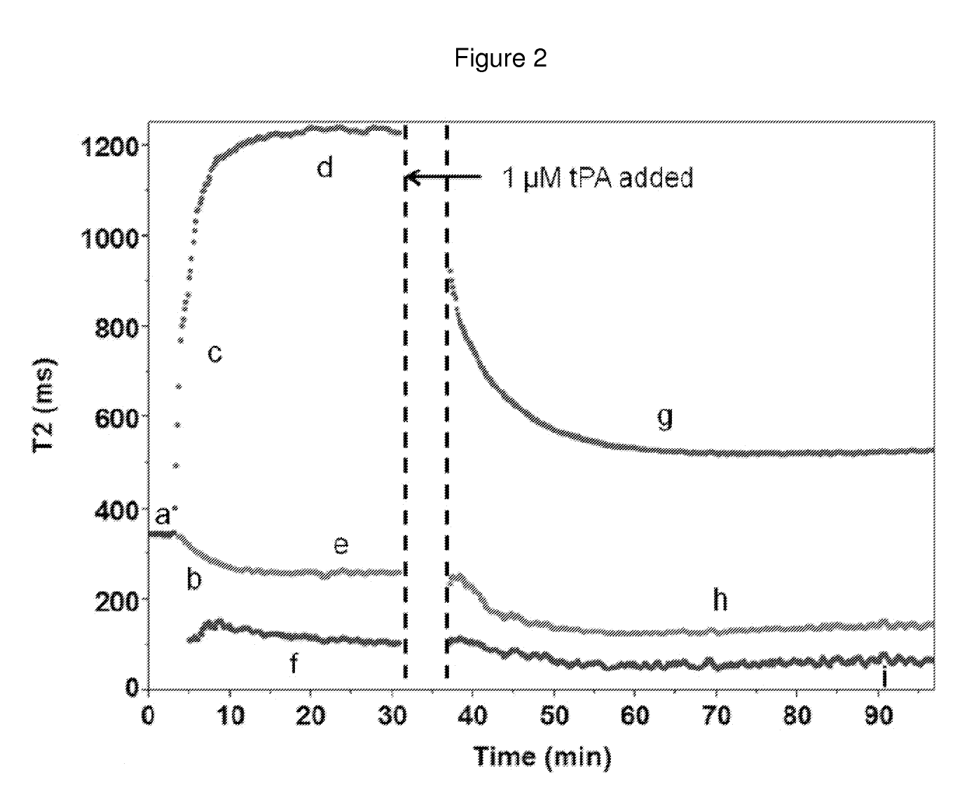

We measured the dependencies of the T2MR signals during clotting of re-calcified citrated blood samples from healthy donors initiated by adding 3 U/ml thrombin. Thrombin activates platelets and cleaves fibrinogen to form a three-dimensional fibrin network stabilized by factor XIIIa. Addition of thrombin led to rapid formation of a gelatinous meshwork that filled the sample volume accompanied by a small, rapid decrease in the T2MR signal over tens of seconds due to the sample transitioning from a liquid to gel state. In the initial gel state, only one relaxation rate was observed (FIG. 2, part a), reflecting uniform distribution of erythrocytes and other blood components. Approximately four minutes after thrombin addition, the T2MR signal split into two peaks representing distinct water populations in slow exchange with each other. One peak decreased in T2 value (FIG. 2, part b), indicating increasing erythrocyte concentration in one compartment, while the T2 value of the other peak increased rapidly, consistent with depletion of erythrocytes (FIG. 2, part c). Approximately 20 minutes after addition of thrombin, the upper peak reached a plateau (FIG. 2, part d). The lower peak at ˜300 ms decreased in T2 value, associated with visible clot contraction, until around 10 minutes when it reached a plateau at ˜275 ms (FIG. 2, part e). A third peak first appeared at 6 minutes at a lower T2 value (˜100 ms) (FIG. 2, part f).

We then assessed the sensitivity of the T2MR platform to fibrinolysis by adding tissue-type plasminogen activator (tPA) to the clotted samples 30 minutes after thrombin. After tPA addition, the T2 value of the upper peak decreased rapidly (FIG. 2, part g), the middle peak decreased from 250 to 175 ms (FIG. 2, part h), while the third peak at ˜100 ms persisted (FIG. 2, part i).

### Example 3

### Analyzing Isolated Sample Components

The T2 values of individual components of blood were determined using samples fractionated as described above or using clotted whole blood components. All samples were pre-warmed at 37° C. for 1 min before transferring to a T2MR reader for measurement. For plasma, 40 μL of PPP was measured. For serum, 200 μL of whole blood was clotted by addition of 2 U/ml thrombin to re-calcified blood. After a 30 min incubation at 37° C., the tube was centrifuged for 1 min at 10,000 g and 40 μL of the upper (serum) fraction was measured. To measure isolated retracted clots, re-calcified blood was allowed to clot for 1 hr following addition of 2 U/ml thrombin at 37° C. Then, erythrocytes excluded from clot were removed by washing the clot with 100 μL of PPP by gentle pipetting. The liquid was aspirated and disposed. This washing protocol was repeated two more times. To measure the isolated clot, all liquid was aspirated after the washing steps.

To interpret the T2MR signals during clot formation, contraction, and lysis, the major biological components of the system were measured in isolation and upon recombination (Table 1). Consistent with relaxation theory, T2MR signals were highest for serum, intermediate for plasma and lowest for whole blood and contracted clots. The range of T2 values for whole blood from healthy donors, 400-285 ms, corresponds to hematocrit values of 35%-55% and the range for reconstituted samples, 575-189 ms, corresponds to hematocrit values of 21%-83%. The higher T2MR signals in serum relative to whole blood arise from the lack of erythrocytes (and associated hemoglobin), which accelerates relaxation of water protons. The T2MR signal of plasma is lower than that of serum due to the relatively higher concentration of proteins that increase relaxation rates by exchange between free and protein bound water (Table 1).

TABLE 1
T2 values of isolated components of clotted blood
for N = 6 donor samples.

| Isolated | |
|---|---|
| Component | T2 (ms) |
| Serum | 1000-1200 |
| Plasma | 800-1000 |
| Homogenous whole | 400-285 for 35-55% |
| blood | hematocrit |
| Loosely bound or | 200-300 |
| unbound erythrocytes | |
| Contracted clot | 75-175 |

We next measured the T2MR signals of isolated contracted clots. One hour after re-calcified citrated whole blood was clotted with 2 U/ml thrombin, contracted clots were removed, washed with platelet poor plasma and T2MR signals were measured. Clots remained intact during manipulation indicating tight contraction. The T2MR signals generated by isolated clots ranged from 100-150 ms (n=6), consistent with this signal arising from a tightly contracted clot with a hematocrit approaching 100% based on equation 10.

$$T {2}_{o} = {( \frac{{X}_{e}}{T {2}_{e}} + \frac{{X}_{p}}{T {2}_{p}} )}^{- 1} \qquad ( 10 )$$

where T2₀ is the observed T2 value, T2, and T2ₚ are the intrinsic relaxation time constants for the erythrocyte and plasma compartments, and Xₑ and Xₚ are the mole fraction of total water in each compartment.

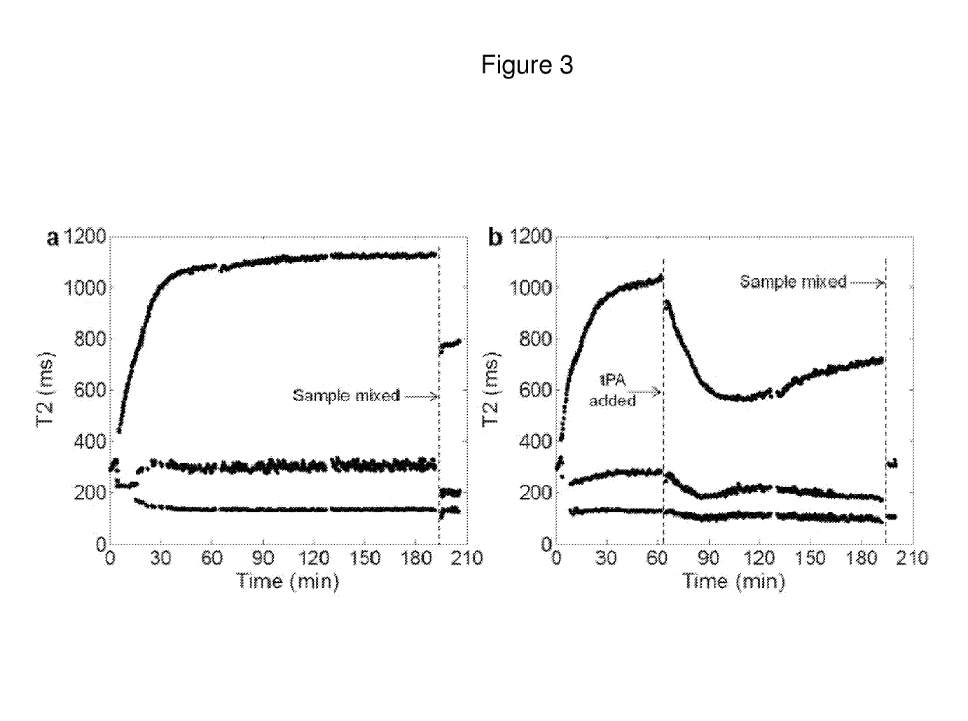

The compartment generating the signal in FIG. 2, part b, at 300 ms that dropped to 200 ms was assessed by testing two conditions: (1) re-calcified citrated whole blood activated with thrombin to form a contracted clot and (2) re-calcified citrated whole blood activated with thrombin followed by addition of tPA. After incubation, samples were analyzed before and after mixing with a pipette to re-suspend unbound erythrocytes. In the sample clotted with thrombin, the 200-300 ms signal remained after mixing, but the T2 value of both the upper peak and this peak decreased as some unbound erythrocytes were dislodged by mixing (FIG. 3a). In the sample clotted with thrombin then lysed with tPA, the 200-300 ms signal disappeared altogether after mixing. The upper T2 peak decreased in T2 value as the erythrocytes that were released from the fibrin network during clot lysis were resuspended by mixing (FIG. 3b). These data support the conclusion that the T2MR signal at 200-300 ms originates from erythrocytes loosely bound to platelets and fibrin that is susceptible to tPA-induced fibrinolysis. The observation that the lowest T2MR signal in clotted samples persists after tPA addition is consistent with the signal emanating from a tightly compacted clot resistant to fibrinolysis.

### Example 4

### Clotting Reconstituted Samples with Calcium and Kaolin

The combined effect of hematocrit and platelet count on the T2MR signal was explored by generated 96 reconstituted samples of varying hematocrit and platelet count. These samples were prepared as described previously. The clotting experiments were performed by mixing 34 μL of reconstituted blood, 2 μL 0.2 M CaCl₂, and 2 μL kaolin solution (Haemonetics, Braintree, Mass.). All reagents were pre-warmed at 37° C. prior to T2MR measurement.

### Example 5

### T2 Relaxation and Hematocrit

A single T2 value was observed for the measurement of unclotted blood, consistent with previous studies with similar magnetic fields and short inter-echo CPMG delays. The dependence of T2 relaxation on the blood oxygenation state at higher fields and much longer echo times (tens of ms) has been successfully used for in vivo MRI. The diminished dependence of blood oxygenation state on T2 relaxation at low magnetic fields and short echo times (hundreds of microseconds) has been previously studied and suggests the difference between oxygenated and deoxygenated blood to be <25 ms under our measurement conditions. Further experiments and optimization will be necessary to fully characterize this dependence. T2MR signal dependence on hematocrit can be modeled by equation 10 (above), where T2ₒ is the observed T2 value, T2ₑ and T2ₚ are the intrinsic relaxation time constants for the erythrocyte and plasma compartments, and Xₑ and Xₚ are the mole fraction of total water in each compartment. Measured data were fitted best when T2ₚ=1000 ms and T2ₑ=165 ms.

### Example 6

### Prothrombin Time Method Comparison

A T2MR citrated blood prothrombin time (PT) assay was developed using Innovin® as a reagent and measuring the time at which the T2MR signal changed due to clot formation. Dade® Innovin® (Siemens Healthcare Diagnostics, Newark, Del.) was prepared according to manufacturer instructions. A stock solution of fibrinogen (60 mg/ml) was prepared in saline. To measure the clotting time using T2MR, 150 μL of citrated blood was mixed with 2.6 μL of the fibrinogen solution. All components were incubated for 2 minutes at 37° C. prior to T2MR measurements. Blood and fibrinogen (40 μL) was positive pipetted into the 20 μl of Innovin® and the T2MR readings were initiated immediately. T2 values were collected at a sampling rate of 2 sec for 2 min. The resulting T2 vs. time data was fit with a 5 parameter logistic, and the clotting time was calculated using the “half maximal effective dose” (EC50) equation commonly used to determine the potency of drugs when concentration is plotted versus time instead of T2 value. The reference method clotting time was obtained by running the same samples on the Stago ST4 system using PRP following the manufacturer's protocol.

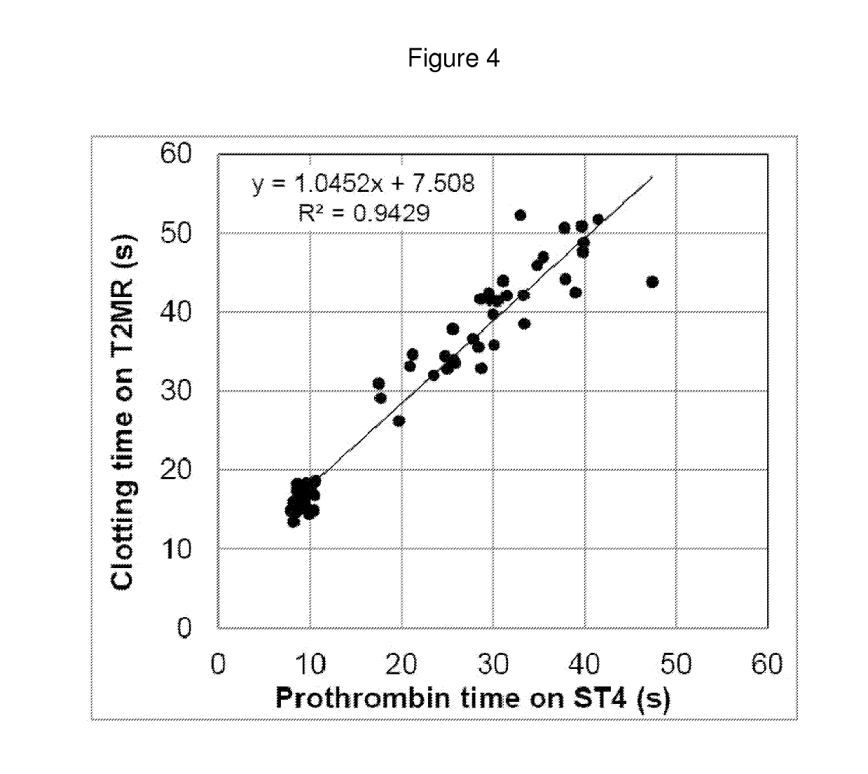

A T2MR citrated blood prothrombin time (PT) assay was developed using Innovin® as a reagent and measuring the time at which the T2MR signal changed due to clot formation. The 2:1 sample to reagent dilution used in this assay formulation necessitated the addition of a fibrinogen reagent to ensure adequate changes in the T2MR signal upon clotting. This increased the robustness and precision of the assay, while still producing PT times that correlated well with the reference method. Fibrinogen was not added for other assays where sample dilution was less. The T2MR PT assay gave % CV=3.5% for 10 replicates across 23 donor samples (Table 2) and a correlation of R²=0.94 over 68 donor samples from normal and anti-coagulated donors when compared with measurement in plasma using the Stago ST4 system (FIG. 4).

TABLE 2
Precision of PT measurements using T2MR.

| | Average T2MR PT | T2MR % CV |
|---|---|---|
| Sample | n = 10 (sec) | n = 10 |
| 1 | 16.0 | 2.6% |
| 2 | 14.5 | 5.2% |
| 3 | 13.8 | 3.8% |
| 4 | 17.0 | 4.3% |
| 5 | 15.2 | 2.7% |
| 6 | 16.3 | 2.2% |
| 7 | 14.5 | 4.0% |
| 8 | 17.9 | 4.5% |
| 9 | 17.0 | 4.1% |
| 10 | 15.9 | 2.8% |
| 11 | 44.9 | 4.3% |
| 12 | 32.2 | 1.9% |
| 13 | 53.1 | 3.6% |
| 14 | 36.9 | 2.8% |
| 15 | 50.9 | 3.8% |
| 16 | 41.1 | 3.6% |
| 17 | 47.3 | 2.5% |
| 18 | 41.9 | 2.5% |
| 19 | 36.2 | 5.0% |
| 20 | 36.6 | 2.9% |
| 21 | 42.6 | 2.0% |
| 22 | 31.2 | 4.9% |
| 23 | 44.6 | 4.9% |

### Example 7

### Measurement of Clot Strength

To demonstrate correlation of T2MR to the thromboelastography maximum amplitude (TEG MA) parameter, citrated blood samples were titrated with abciximab (ReoPro, Eli Lilly and Company, Indianapolis, Ind.), an inhibitor of the platelet glycoprotein allbβ3 receptor that binds fibrin and is essential for clot contraction. A 0.5 mg/ml solution of abciximab was prepared by diluting the stock 10 mg/5 ml solution by 1:4 in saline. The abciximab-treated blood samples were incubated for at least 5 min prior to clotting. Clotting was initiated by adding 2 μL 0.2 M CaCl₂ and 2 μL TEG kaolin to 34 μL of abciximab-treated blood sample. To compare the T2MR signal to TEG MA, a ΔT2 parameter was calculated by taking the difference in T2 between the upper and middle peaks at a time point 13 min after adding calcium and kaolin. The TEG MA values were measured on the same samples following manufacturer instructions.

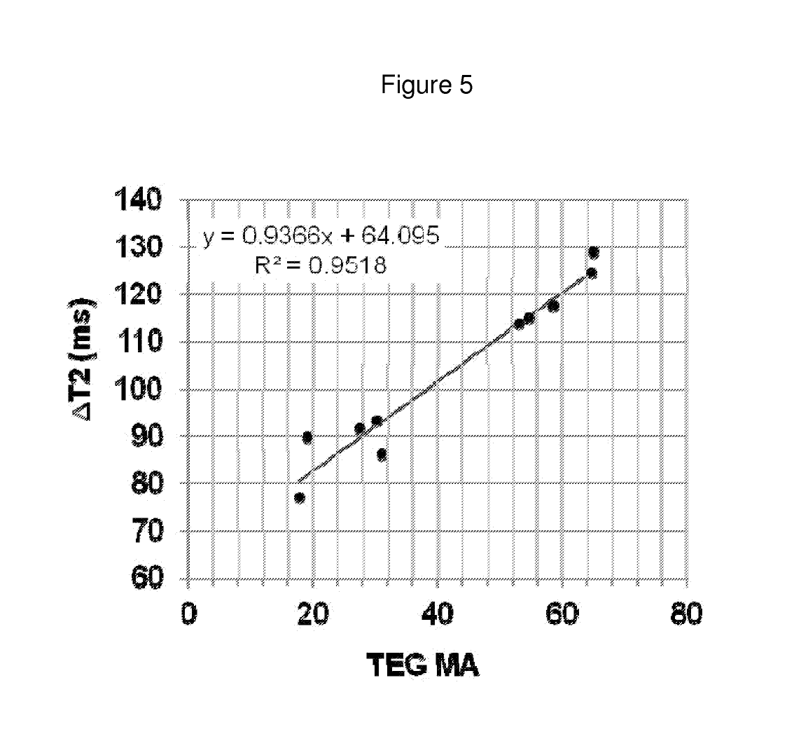

For comparison between T2MR and TEG, calcium kaolin activation of citrated blood was used and normal donor samples were treated with various amounts of abciximab, an inhibitor of the platelet glycoprotein allbβ3 receptor that binds fibrin and is essential for clot contraction. The difference in T2 value between the peaks associated with serum and loosely compacted clot showed a strong correlation (R2=0.95) with the TEG MA values across 10 samples from 3 donors at varying amounts of added abciximab (FIG. 5).

### Example 8

### Measurement of Platelet Activity

To isolate the T2MR signal response to platelet activity stimulated by adenosine diphosphate (ADP), we used a reagent mix containing final concentrations of 10 mM CaCl₂, 20 U/ml heparin to inhibit thrombin, and 1/38 dilution of the standard preparation of Activator F, a proprietary mix of reptilase and factor XIIIa (Haemonetics, Braintree, Mass.), to quickly generate a fibrin network, and 5 μM ADP. To perform the test, 34 μL of citrated blood with or without 100 μM 2-methylthioadenosine 5′-monophosphate (2-MeSAMP) was added to 4 μL of activation reagent in a PCR tube and T2MR signals were monitored for 10 min. The platelet function of PRP with platelet count matched to that of whole blood from the same samples was tested concurrently with LTA on a Chrono-log optical aggregometer. To assess correlation, we used the criteria of maximum percent aggregation by LTA over 6 min and the maximum percent change in T2MR signal of the upper peak over 10 min. The cutoffs for a positive result were 55% signal change for LTA and 100% signal change for T2MR.

Whereas measurement of platelet function by light transmission aggregometry (LTA) measures platelet-platelet interactions, T2MR measures platelet function via platelet-mediated clot contraction, an integrated activity that includes platelet activation, aggregation, adhesion to the clot, and cell-mediated contraction. To demonstrate the configurability of the T2MR platform for platelet function assays, we compared T2MR with citrated blood and LTA performed with platelet rich plasma using adenosine diphosphate (ADP) as a platelet activator. To isolate the signal response to platelet activation, we used a reagent mix containing ADP, heparin to inhibit thrombin, and reptilase and factor XIIIa to quickly generate a fibrin network. We compared T2MR with LTA across samples tested with ADP in the presence and absence of the inhibitor 2-methylthioadenosine 5′-monophosphate (2-MeSAMP). Positive agreement between T2MR and LTA for 20 samples was 100%, and negative agreement over 8 samples was 75% (6/8), giving an overall agreement of 93% (26/28) (Table 3).

TABLE 3
Contingency table comparing ADP platelet activity
measurements on T2MR and LTA.

LTA

| | | Yes | No | Totals |
|---|---|---|---|---|
| T2MR | Yes | 20 | 2 | 22 |
| | No | 0 | 6 | 6 |
| | Totals | 20 | 8 | 28 |

### Example 9

### Correlation of T2MR with Other Diagnostic Tests

While T2MR can be used to obtain new insights into the physical states of microenvironments within blood samples, it can also be configured to measure standard hemostasis parameters. To demonstrate this, we performed method comparison studies against the Sysmex pocH-100i hematology analyzer for hematocrit where an R²=0.95 for 40 donor samples and an average precision of % CV=4.8% for N=10 replicates across 13 donor samples; for prothrombin time (PT) against the Stago ST4 system, where a correlation of R²=0.94 over 68 donor samples from normal and anti-coagulated donors and a % CV=3.5% for N=10 replicates across 23 donor samples was observed; for thromboelastography (TEG) clot strength, a correlation of R²=0.95 between T2MR and TEG MA values across 10 samples was observed; and for platelet function measurements an overall agreement of 93% was observed between T2MR and light transmission aggregometry (LTA) for activation by ADP.

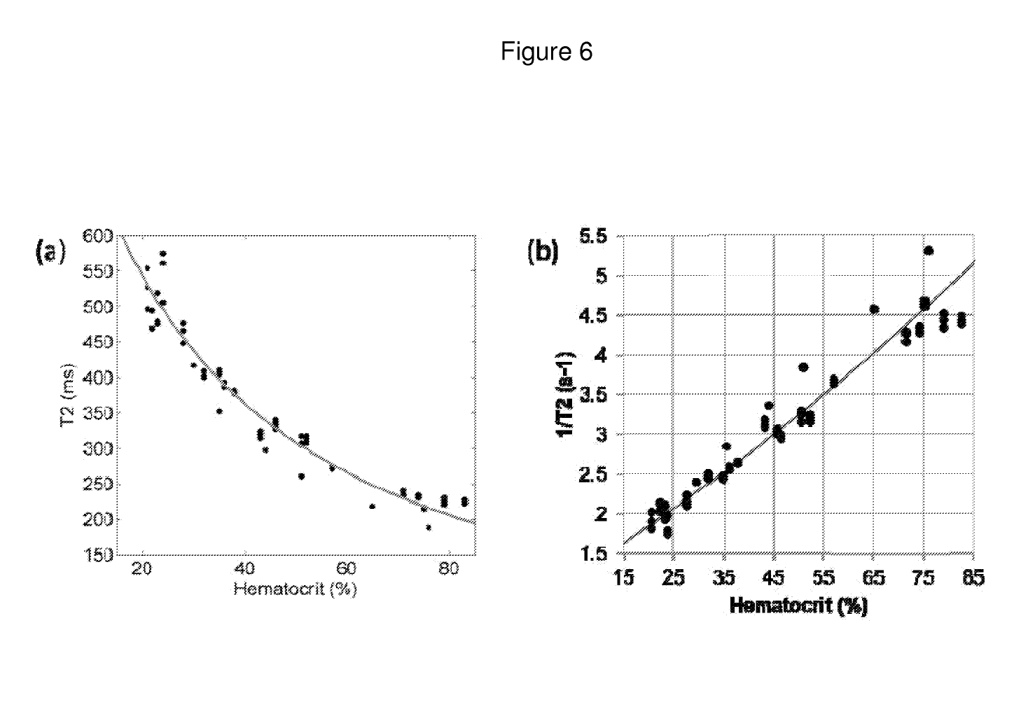

Hematocrit measurements can also be performed via T2MR. A method comparison study between T2MR and the Sysmex pocH-100i hematology analyzer for determining hematocrit revealed high levels of correlation. Samples were generated from reconstituted blood from 40 independent donors in Table 4. A T2MR value was measured for each sample and converted to hematocrit using the calibration curves shown in FIGS. 6a and 6b, and the hematocrit was measured on the Sysmex platform. The two methods correlated with R²=0.95. T2MR hematocrit measurements also show a great deal of precision. Data in Table 5 depict T2MR values collected for 10 repetitions from each of 13 independent donor samples and converted to hematocrit values using the calibration curves shown in FIGS. 6a and 6b. The average % CV was 4.8%.

A T2MR citrated blood prothrombin time (PT) assay was developed using Innovin as a reagent and measuring the time at which the T2MR signal changed due to clot formation. The 2:1 sample to reagent dilution used in this assay formulation necessitated the addition of a fibrinogen reagent to ensure adequate changes in the T2MR signal upon clotting. This increased the robustness and precision of the assay, while still producing PT times that correlated well with the reference method. Fibrinogen was not added for other assays where sample dilution was less. The T2MR PT assay gave % CV=3.5% for 10 replicates across 23 donor samples (Table 2) and a correlation of R²=0.94 over 68 donor samples from normal and anti-coagulated donors when compared with measurement in plasma using the Stago ST4 system (FIG. 4).

For comparison between T2MR and TEG, calcium kaolin activation of citrated blood was used and normal donor samples were spiked with various amounts of abciximab, an inhibitor of the platelet glycoprotein allbβ3 receptor that binds fibrin and is essential for clot contraction. The difference in T2 value between the peaks associated with serum and loosely compacted clot showed a strong correlation (R2=0.95) with the TEG MA values across 10 samples from 3 donors at varying amounts of added Abciximab (FIG. 5).

Whereas measurement of platelet function by light transmission aggregometry (LTA) measures platelet-platelet interactions, T2MR measures platelet function via platelet-mediated clot contraction, an integrated activity that includes platelet activation, aggregation, adhesion to the clot, and cell-mediated contraction. To demonstrate the configurability of the T2MR platform for platelet function assays, we compared T2MR with citrated blood and LTA performed with platelet rich plasma using adenosine diphosphate (ADP) as a platelet activator. To isolate the signal response to platelet activation, we used a reagent mix containing ADP, heparin to inhibit thrombin, and reptilase and factor XIIIa to quickly generate a fibrin network. We compared T2MR with LTA across samples tested with ADP in the presence and absence of the inhibitor 2-methylthioadenosine 5′-monophosphate (2-MeSAMP). Positive agreement between T2MR and LTA for 20 samples was 100%, and negative agreement over 8 samples was 75% (6/8), giving an overall agreement of 93% (26/28) (Table 3).

TABLE 4
Method comparison for hematocrit measurement on T2MR
and Sysmex pocH-100i hematology analyzer.

| | T2MR | Sysmex |
|---|---|---|
| | hematocrit | hematocrit |
| Sample # | (%) | (%) |
| 1 | 43.7 | 22.3 |
| 2 | 38.1 | 20.0 |
| 3 | 34.3 | 18.7 |
| 4 | 54.5 | 29.2 |
| 5 | 23.4 | 14.2 |
| 6 | 39.0 | 22.5 |
| 7 | 58.3 | 32.6 |
| 8 | 28.9 | 18.4 |
| 9 | 33.2 | 21.1 |
| 10 | 67.0 | 38.5 |
| 11 | 28.2 | 19.6 |

TABLE 5
Precision of hematocrit measurements on T2MR.

| | Average T2MR | % CV |
|---|---|---|
| Sample # | hematocrit (%) | (10 reps) |
| 1 | 38.5 | 5.0 |
| 2 | 43.0 | 3.7 |
| 3 | 41.8 | 6.6 |
| 4 | 35.4 | 3.2 |
| 5 | 40.7 | 4.2 |
| 6 | 30.0 | 5.0 |
| 7 | 32.7 | 5.2 |
| 8 | 46.7 | 4.5 |
| 9 | 35.8 | 4.5 |
| 10 | 37.3 | 3.4 |
| 11 | 39.3 | 7.6 |
| 12 | 39.6 | 4.5 |
| 13 | 46.0 | 4.6 |

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method for monitoring a clotting process in a whole blood sample comprising:
(a) providing uncoagulated whole blood, fibrinogen, and a clotting activation reagent;
(b) combining the fibrinogen, the clotting activation reagent, and the uncoagulated whole blood to form a reaction mixture that comprises from 50% (v/v) to 90% (v/v) whole blood and a fibrinogen concentration greater than or equal to about 0.5 mg/mL;
(c) making a series of magnetic resonance relaxation rate measurements of water in the reaction mixture; and
(d) on the basis of the results of step (c), determining the clotting time.

2. A method for monitoring a clotting process in a platelet rich plasma sample comprising:
(a) providing uncoagulated platelet rich plasma, fibrinogen, and a clotting activation reagent;
(b) combining the fibrinogen, the clotting activation reagent, and the uncoagulated platelet rich plasma to form a reaction mixture that comprises from 50% (v/v) to 90% (v/v) platelet rich plasma and a fibrinogen concentration greater than or equal to about 0.5 mg/mL;
(c) making a series of magnetic resonance relaxation rate measurements of water in the reaction mixture; and
(d) on the basis of the results of step (c), determining the clotting time.

3. A method for monitoring a clotting process in a platelet poor plasma sample comprising:
(a) providing uncoagulated platelet poor plasma, fibrinogen, and a clotting activation reagent;
(b) combining the fibrinogen, the clotting activation reagent, and the uncoagulated platelet poor plasma to form a reaction mixture that comprises from 50% (v/v) to 90% (v/v) platelet poor plasma and a fibrinogen concentration greater than or equal to about 0.5 mg/mL;
(c) making a series of magnetic resonance relaxation rate measurements of water in the reaction mixture; and
(d) on the basis of the results of step (c), determining the clotting time.

4. The method of any one of claims 1-3, wherein said clotting activation reagent is selected from RF, AA, ADP, CK, TRAP, epinephrine, collagen, tissue factor, celite, ellagic acid, and thrombin.

5. The method of any one of claims 1-4, further comprising repeating steps (a)-(d) to produce a replicate value of the clotting time.

6. The method of any one of claims 1-4, wherein the fibrinogen concentration in the reaction mixture is sufficient to produce a clotting time having coefficient of variation of less than 7% when the clotting time is measured at least 10 times.

7. The method of any one of claims 1-6, wherein step (c) comprises making a series of magnetic resonance relaxation rate measurements of water in the reaction mixture within a sample tube, wherein the inner surface of the sample tube controls fibrin adhesion.

8. The method of any one of claims 1-7, wherein step (c) comprises (i) making a plurality of T2 relaxation rate measurements of water in the reaction mixture to produce a plurality of decay curves, and (ii) calculating from said plurality of decay curves a plurality of T2 relaxation spectra.

9. A method of evaluating a blood sample from a subject comprising (i) performing the method of any one of claims 1-8 on the blood sample, or an extract thereof, to determine the clotting time; and (ii) on the basis of step (i), determining whether the subject is hypercoagulable, hypocoagulable, or normal.

10. A method of evaluating a blood sample from a subject comprising (i) performing the method of any one of claims 1-8 on the blood sample, or an extract thereof, to determine the clotting time; and (ii) on the basis of step (i), determining whether the subject is at risk of thrombotic complications or the subject is resistant to antiplatelet therapy.

11. A method of evaluating a blood sample from a subject comprising (i) performing the method of any one of claims 1-8 on the blood sample, or an extract thereof, to determine the clotting time; and (ii) on the basis of step (i), determining whether the subject has a coagulopathy.

12. The method of any one of claims 1-11, wherein step (c) comprises making a series of magnetic resonance relaxation rate measurements of water in the reaction mixture within a sample tube having a total volume of from 30 to 60 μL.

13. A method for monitoring a clotting process in a whole blood sample comprising:
(a) providing uncoagulated whole blood and a clotting activation reagent;
(b) combining the clotting activation reagent and the uncoagulated whole blood in a sample tube to form a reaction mixture that comprises from 50% (v/v) to 90% (v/v) whole blood and a total volume of from 30 to 60 μL;
(c) making a series of magnetic resonance relaxation rate measurements of water in the sample tube; and
(d) on the basis of the results of step (c), determining the clotting time.

14. A method for monitoring a clotting process in a platelet rich plasma sample comprising:
(a) providing uncoagulated platelet rich plasma and a clotting activation reagent;
(b) combining the clotting activation reagent and the uncoagulated platelet rich plasma in a sample tube to form a reaction mixture that comprises from 50% (v/v) to 90% (v/v) platelet rich plasma and a total volume of from 30 to 60 μL;
(c) making a series of magnetic resonance relaxation rate measurements of water in the sample tube; and
(d) on the basis of the results of step (c), determining the clotting time.

15. A method for monitoring a clotting process in a platelet poor plasma sample comprising:
(a) providing uncoagulated platelet poor plasma and a clotting activation reagent;
(b) combining the clotting activation reagent and the uncoagulated platelet poor plasma in a sample tube to form a reaction mixture that comprises from 50% (v/v) to 90% (v/v) platelet poor plasma and a total volume of from 30 to 60 μL;
(c) making a series of magnetic resonance relaxation rate measurements of water in the sample tube; and
(d) on the basis of the results of step (c), determining the clotting time.

16. The method of any one of claims 13-15, wherein said clotting activation reagent is selected from RF, AA, ADP, CK, TRAP, epinephrine, collagen, tissue factor, celite, ellagic acid, and thrombin.

17. The method of any one of claims 13-16, wherein the sample tube has an inner surface that controls fibrin adhesion.

18. The method of any one of claims 13-17, wherein step (c) comprises (i) making a plurality of T2 relaxation rate measurements of water in the sample tube to produce a plurality of decay curves, and (ii) calculating from said plurality of decay curves a plurality of T2 relaxation spectra.

19. A method of evaluating a blood sample from a subject comprising (i) performing the method of any one of claims 13-17 on the blood sample, or an extract thereof, to determine the clotting time; and (ii) on the basis of step (i), determining whether the subject is hypercoagulable, hypocoagulable, or normal.

20. A method of evaluating a blood sample from a subject comprising (i) performing the method of any one of claims 13-17 on the blood sample, or an extract thereof, to determine the clotting time; and (ii) on the basis of step (i), determining whether the subject is at risk of thrombotic complications or the subject is resistant to antiplatelet therapy.

21. A method of evaluating a blood sample from a subject comprising (i) performing the method of any one of claims 13-17 on the blood sample, or an extract thereof, to determine the clotting time; and (ii) on the basis of step (i), determining whether the subject has a coagulopathy.

22. The method of any one of claims 1-8 and 13-18, wherein step (c) further comprises determining the fibrinogen level of the blood sample.

23. The method of any one of claims 1-8 and 13-18, wherein step (c) further comprises determining the hematocrit of the blood sample, wherein the blood sample is a whole blood sample.

24. The method of any one of claims 1-8 and 13-18, wherein step (c) further comprises determining the platelet activity of the blood sample, wherein the blood sample is a whole blood sample or platelet rich plasma.
